(19) United States
(12) Patent Application Publication
(10) Pub. No.: US 2014/0288010 A1
(43) Pub. Date: Sep. 25, 2014

(54) Compositions and Methods for Increasing Stem Cell Survival

(71) Applicant: Georgia Regents Research Institute, Inc., Augusta, GA (US)

(72) Inventors: William D. Hill, Augusta, GA (US); Samuel Herberg, North Augusta, SC (US); Sudharsan Periyasamy-Thandavan, Martinez, GA (US)

(73) Assignee: Georgia Regents Research Institute, Inc., Augusta, GA (US)

(21) Appl. No.: 14/188,340

(22) Filed: Feb. 24, 2014

Related U.S. Application Data
(60) Provisional application No. 61/768,264, filed on Feb. 22, 2013.

(51) Int. Cl. A61K 38/19; A61K 31/155

(52) U.S. Cl. CPC A61K 38/195; A61K 31/155
USPC 514/21.3; 514/635; 435/375

(57) ABSTRACT

Compositions and methods of increasing autophagy in a cell, or population of cells are disclosed. The methods generally include contacting the cell or cells with an effective amount of an agent that increases the bioavailability of an active isoform of SDF-1. Exemplary agents include active SDF-1 polypeptides, metformin, and DPP4 inhibitors. The methods can include administering the agent to a subject, or treating cells in vivo, in vitro or ex vivo. In some embodiments, cells are treated ex vivo and then transplanted into a subject. In a preferred embodiment, the cells are mesenchymal stem cells such as those found in bone marrow. The compositions and methods can be utilized in a number of therapeutic applications including increasing the longevity or survival of a graft or transplant, increasing the rate of recovery from an injury, reducing one or more symptoms of a chronic inflammatory disease and reducing effect associated with aging.

### CROSS REFERENCE TO RELATED APPLICATIONS

This application claims the benefit of and priority to U.S. Provisional Application No. 61/768,264, filed Feb. 22, 2013.

### STATEMENT REGARDING FEDERALLY SPONSORED RESEARCH OR DEVELOPMENT

This invention was made with government support under Agreement P01-AG036675-01 awarded by the National Institutes of Health. The government has certain rights in the invention.

### REFERENCE TO SEQUENCE LISTING

The Sequence Listing submitted Feb. 24, 2014 as a text file named “GRU_{—}2013_{—}026_ST25.txt,” created on Feb. 24, 2014, and having a size of 10,701 bytes is hereby incorporated by reference pursuant to 37 C.F.R. §1.52(e)(5).

### FIELD OF THE INVENTION

The inventions generally relates to methods of increasing stem cell survival by modulating SDF-1.

### BACKGROUND OF THE INVENTION

Over the last decade, numerous studies have revealed that bone marrow-derived mesenchymal stem/stromal cells (BMSCs) hold great potential for cell-based therapy as BMSCs possess multi-lineage potential (Caplan, J. Pathol., 217:318-324 (2009)). For instance, both autologous and allogeneic BMSCs have been utilized to repair or regenerate bone in experimental and clinical studies (Korbling, et al., New England Journal of Medicine, 349:570-582 (2003); Marcacci, et al., Tissue Engineering, 13:947-955 (2007)). However, attempts to transplant BMSCs from whole bone marrow (BM), enriched peripheral blood, or highly purified low-passage cultures almost universally fail to significantly engraft within the BM when infused into the peripheral circulation of animal and human subjects, in large part due to the poor survival of donor cells (Hu, et al., Proc. Natl. Acad. Sci. USA, 96:7294-7299 (1999); Nilsson, et al., Journal of Experimental Medicine, 189:729-734 (1999); Pereira, et al., Proc. Natl. Acad. Sci. USA, 95:1142-1147 (1998); Dominiici, et al., Proc. Natl. Acad. Sci. USA, 101:11761-11766 (2004); Horwitz, et al., Proc. Natl. Acad. Sci. USA, 99:8932-8937 (2002)). After being transplanted, BMSCs can face a complex hostile environment with factors that may promote cell loss/death including inflammatory reactions, hypoxia, oxidative stress including reactive oxygen species, and nutrient starvation.

It is also known that survival of stem and progenitor populations with age, or following survival challenges, may be reduced, and it is believed that loss of stem cells, including “adult” stem cells in stem cell niches and tissue progenitor/stem cells can result in age-associated declines in tissue maintenance and repair. Indeed, loss of stem cells may in-part underlie numerous elements related to a decline in tissue repair capacity throughout the body in aging.

Despite the great therapeutic potential for stem cells in treating acute and chronic diseases, there remains a need to increase the survivability of stem cells to boost their therapeutic efficacy.

Accordingly, it is an object of the invention to provide compositions and methods of increasing the survivability of stem cells in vivo.

It is a further object of the invention to provide compositions and methods for increasing autophagy, reducing apoptosis or a combination thereof of stems cells in vivo.

It is also an object of the invention to provide methods and compositions for increasing the survivability of transplants or grafts in a subject.

It is another object of the invention to provide compositions and methods for increasing recovery from injury or improving injury repair, particularly acute injuries such as trauma, fractures, and defects in a subject.

It is also an object of the invention to reduce the effects of aging, particular the effects of aging on stem cells.

It is a further object of the invention to reduce age-associated declines in tissue maintenance and repair.

### SUMMARY OF THE INVENTION

Compositions and methods of increasing autophagy in a cell, or population of cells are disclosed. The methods generally include contacting the cell or cells with an effective amount of an agent that increases the bioavailability of an active isoform of SDF-1. Agents that increase the bioavailability of an active isoform of SDF-1 can be polypeptides including active isoforms of SDF-1, metformin, transcription factors that increase expression of SDF-1, and functional nucleic acids and other inhibitors that reduce or inhibit the ability of an miRNAs such as miRs 29a-5p, 1244, 141, 144, 200a, or 200c from targeting SDF-1 mRNA.

An agent that increases the bioavailability of an active isoform of SDF-1 can also be an agent that decreases expression or production of inactive or antagonistic forms of SDF-1, such as an inhibitor of a metalloproteinase, CD26/dipeptidyl peptidase IV (DPP4), a serine protease, or a leukocyte elastase. Inhibitors of DPP4 include sitagliptin, vildagliptin, saxagliptin, linagliptin, dutogliptin, gemigliptin, alogliptin, and pharmaceutically acceptable salts, or active analogs thereof, and functional nucleic acids that target DPP4 mRNA, such as the miRNA miR-3173-5p.

An agent that increases the bioavailability of an active isoform of SDF-1 can also be an agent that increases expression of an SDF-1 receptor. SDF-1 receptors include CXCR4 and CXCR7.

The agent can be a pharmaceutical composition including a carrier suitable for administration to a subject. The pharmaceutical composition can include two or more agents that increase the bioavailability of an active isoform of SDF-1, and optionally one or more additional therapeutic agents.

The disclosed methods can include administering the agent that increases autophagy of a cell to a subject, or treating cells in vitro or ex vivo. For in vivo applications, the agent can be administered systemically or locally to the site to be treated.

In some embodiments, cells are treated ex vivo and then administered to or transplanted into a subject. The cells can be isolated from the subject prior to contacting the cells with the agent.

The cells can be, for example, progenitor cells, multipotent cells, pluripotent cells, embryonic stem cells, inner mass cells, bone marrow stem cells, cells from umbilical cord blood, and ectoderm, mesoderm, or endoderm, for cells derived therefrom. The cells can be adult stem cells such as hematopoietic stem cells, mesenchymal stem cells, epithelial stem cells, muscle satellite cells, or induce pluripotent stem (iPS) cells. In a preferred embodiment, the cells are mesenchymal stem cells such as those found in bone marrow.

The compositions and methods can be utilized in a number of therapeutic applications. For example, the compositions and methods can be used to increase the longevity or survival of a graft or transplant, increase recovery from an injury such as those resulting from trauma, wounds, fractures, defects, or surgery.

The compositions and methods can also be utilized to treat chronic injuries or diseases including inflammatory diseases such as inflammatory joint disease (i.e., rheumatoid arthritis), inflammatory bowel disease (e.g. Crohn's disease or ulcerative colitis), coeliac disease, lung inflammation (asthma, chronic obstructive pulmonary disease, alveolitis), renal disease (nephritis, vasculitis) and disease affecting nerve and muscle (myositis, inflammatory neuropathy).

The compositions and methods can also be utilized to one or more symptoms associated with aging such as a decline in tissue maintenance and repair, loss of stem cells, or osteoporosis.

### BRIEF DESCRIPTION OF THE DRAWINGS

FIG. 1 is a line graph showing colorimetric quantification of DMSO-solubilized MTT formazan at 540 nm of isolated BMSC cells: Tet-Off-SDF-1β with and without Dox and Tet-Off-EV controls with and without Dox over time (1, 3, and 7 d, ±100 ng/ml Dox, n=6, 3 independent experiments).

FIGS. 2A and B are bar graphs showing the cell number of isolated BMSC cells: Tet-Off-SDF-1β with and without Dox and H₂O₂ (FIG. 2A) and Tet-Off-EV controls with and without Dox and H₂O₂ (FIG. 2B), with or without trypan blue staining (6 h, ±100 ng/ml Dox, ±1.0 mM H2O2, ***p<0.0001, −Dox; H2O2 vs. +Dox; H2O2, n=3, 3 independent experiments).

FIGS. 3A and 3B are images of autoradiograms of western blots detecting PARP, cleaved PARP, cleaved caspase-3, Beclin-1, LC3B-I, LC3B-II, and beta-actin (loading control) of isolated BMSC cells: Tet-Off-SDF-1β with and without Dox and H₂O₂ (FIG. 3A) and Tet-Off-EV controls with and without Dox and H₂O₂ (FIG. 3B). FIGS. 3C-G are bar graphs showing densitometry quantification of immunoreactive bands of FIGS. 3A-3B: PARP (FIG. 3C), cleaved PARP (FIG. 3D), cleaved caspase-3 (FIG. 3E), Beclin-1 (FIG. 3F), LC3B-II (FIG. 3G) (6 h, ±100 ng/ml Dox, ±1.0 mM H2O2, ***p<0.0001, −Dox; H2O2 vs. +Dox; H2O2, n=3, 3 independent experiments).

FIG. 4 is a bar graph showing the results of qPCR analysis of autophagic mRNA levels in CD271+ human mesenchymal stem cells isolated from “young” and “old” human bone marrow discards. The results show that expression of markers for autophagy including LC3B, p62, and Beclin are reduced in “aged” subjects.

FIG. 5 is a diagram illustrating a proposed role for the SDF-1 signaling pathway in regulating autophagy at the transcriptional and protein level in MSCs.

FIG. 6 is an image of autoradiograms of western blots detecting LC3B, p62, pERK, beta-actin in protein isolated from 18 month MSCs control cells, and cells treated with active SDF-1β, cleaved (c1) inactive C1.SDF-1β for 48 hours.

FIG. 7 is bar graph showing the results of an assay measuring the level of AMPK-alpha1 and LC3B mRNA in 24 month MSCs control cells and cells treated with 100 ng/ml of active SDF-1β for 48 hours. *p<0.05 compared to control group.

FIG. 8 is bar graph showing migration ability of 18 month old MSCs following 6 hours treatment with active SDF-1β, cleaved (c1) inactive C1.SDF-1β, or AMD3100. The data shown are averages of quadruplicate wells. Bar graph represents RFU of migrated cells normalized to vehicle control group +/−SEM. *p<0.05 verse control group.

FIG. 9 is a bar showing mineralization (measured by Alizarin red staining) in MSC monolayers treated with vehicle (control), active SDF-1β, cleaved (c1) inactive C1.SDF-1β, active SDF-1β and cleaved (c1) inactive C1.SDF-1β, metformin, and Compound C relative to undifferentiated control.

FIG. 10 is an image of autoradiograms of western blots detecting LC3B, p62, pERK, beta-actin in protein isolated from 24 month MSCs control cells, and cells treated with active SDF-1β (100 ng/ml), metformin (100 μM), or active SDF-1β and metformin for 48 hours.

FIG. 11 is bar graph showing the results of an assay measuring the level of SDF-1β, CXCR4, and LC3B mRNA in MSCs following treatment with metformin can compared to controls.

FIG. 12 is bar graph showing the results of an assay to measure the ability of 18 month old MSC to migrate toward bone marrow supernatant isolated from 3 month old mice and 18 month old mice with (“normal”) and without AMD3100 incubation for 4 hours.

FIG. 13 is a bar graph showing the relative DPPVI activity of bone marrow supernatant isolated from 3 month old mice and 18 month old mice.

FIG. 14 is a bar graph showing the results of qPCR to measure the mRNA levels of SDF-1α, SDF-1β, CXCR4, CXCR7, BMP2, RUNX2, and OCN, in human CD271+ MSC isolated from young and old human bone marrow discards.

FIGS. 15A-D are bar graphs showing the results of qPCR to measure the mRNA levels of SDF-1α (15A and 15C), SDF-1β (15B and 15C) in controls (15A, 15B, 15C, 15D, left hand bar) and following transfection of murine BMSCs with miRNAs miR-200a (15A and 15B, middle bar), miR-141 (15A and 15B, right hand bar), miR-200c (15C and 15D, middle bar), and miR-144 (15C and 15D, right hand bar).

FIGS. 16A and 16B show SDF-1α (16A), SDF-1β (16B) protein expression and secretion into culture media in murine BMSC control cells and cells transfected with miR-200a or miR-141 (*p<0.001, **p<0.05).

FIGS. 17A and 17D are images of autoradiograms of western blots detecting pErk 1/2, Erk1/2, pSmad 1/5/8, Smad 1/5/8, and beta-actin (loading control) of isolated BMSC cells: Tet-Off-SDF-1β with and without Dox, BMP2, AMD3100, U0126, and combinations thereof (FIG. 17A) and Tet-Off-EV controls with and without Dox, BMP2, AMD3100, U0126, and combinations thereof (FIG. 17D). FIGS. 17B-C and 17E-F are bar graphs showing densitometry quantification of immunoreactive bands of FIGS. 17A and 17D as a ratio of pErk 1/2 to Erk1/2 (17B and 17E) and pSmad 1/5/8 to Smad 1/5/8 (17C and 17F).

### DETAILED DESCRIPTION OF THE INVENTION

### I. Definitions

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences.

As used herein, an “expression control sequence” is a DNA sequence that controls and regulates the transcription and/or translation of another DNA sequence.

The term “polypeptides” includes proteins and fragments thereof. Polypeptides are as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Since it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly when the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations include at least one amino acid deletion, substitution, including conservative and non-conservative substitution, or insertion, wherein the alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from the total number of amino acids in the reference polypeptide.

As used herein, the term “low stringency” refers to conditions that permit a polynucleotide or polypeptide to bind to another substance with little or no sequence specificity.

As used herein, the term “purified” and like terms relate to the isolation of a molecule or compound in a form that is substantially free (at least 60% free, preferably 75% free, and most preferably 90% free) from other components normally associated with the molecule or compound in a native environment. As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “treating” includes alleviating the symptoms associated with a specific disorder or condition and/or preventing or eliminating the symptoms.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “host cell” refers to prokaryotic and eukaryotic cells into which a recombinant vector can be introduced.

As used herein, “transformed” and “transfected” encompass the introduction of a nucleic acid (e.g. a vector) into a cell by a number of techniques known in the art.

As used herein, the phrase that a molecule “specifically binds” or “displays specific binding” to a target refers to a binding reaction which is determinative of the presence of the molecule in the presence of a heterogeneous population of other biologics.

Under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

The terms “individual”, “host”, “subject”, and “patient” are used interchangeably herein, and refer to a mammal, including, but not limited to, humans, rodents, such as mice and rats, and other laboratory animals.

As used herein, the term “cell surface marker” refers to any molecule such as moiety, peptide, protein, carbohydrate, nucleic acid, antibody, antigen, and/or metabolite presented on the surface or in the vicinity of a cell sufficient to identify the cell as unique in either type or state.

The “bioactivity” of one or more isoforms of SDF-1 refers to the biological function of SDF-1 polypeptides. Bioactivity can be increased by increasing the activity of basal levels of active isoforms of SDF-1; increasing the avidity of basal levels of active isoforms of SDF-1; increasing the quantity of active isoforms of SDF-1; increasing the ratio of active isoforms of SDF-1 relative to inactive or antagonistic isoforms of SDF-1, increasing the expression levels of active isoforms of SDF-1, increasing the life-life of active isoforms of SDF-1; decreasing the levels, availability, or life-life of inactive or antagonistic isoforms of SDF-1; increasing the expression or availability of SDF-1 receptor(s); or a combination thereof.

As used herein, the term “active or agonistic forms of SDF-1” refers to isoforms of SDF-1, or fragments, or variants thereof that are capable of mediating signal transduction through the CXCR4 receptor.

As used herein, the term “inactive forms of SDF-1” refers to isoforms of SDF-1, or fragments, or variants thereof that are incapable of mediating signal transduction through the CXCR4 receptor.

As used herein, the term “antagonist forms of SDF-1” refers to isoforms of SDF-1, or fragments, or variants thereof that prevent active forms of SDF-1 from mediating signal transduction through the CXCR4 receptor. Examples of antagonist forms of SDF-1, include, but are not limited to, forms of SDF-1 that bind to the CXCR4 receptor, but do not mediate CXCR4 signal transduction.

### II. Compositions

It has been discovered that autophagy is an important factor in stem cell survival. As discussed in the Examples below, it has been discovered that the SDF-1 (CXCL12) axis can mediate mesenchymal stem cell (MSC) survival by enhancing the autophagic cell pro-survival pathways, and by reducing apoptotic cell death pathways.

MSCs and their descendants are the primary source for SDF-1 in bone, and it has been discovered that both SDF-1, and autophagy levels, change with age in MSCs. Therefore, compositions and methods for increasing cell survival by increasing the bioactivity of SDF-1 in an effective amount to increase autophagy are disclosed. As discussed in more detail below, the compositions typically increase expression or bioactivity of active forms SDF-1 or to reduce or inhibit inactive or antagonistic forms of SDF-1. The compositions can be used in a wide range of therapeutic applications including reducing the effects of aging, increasing the acceptance or survivability of grafts or transplants, increasing the rate of repair or recovery from acute injuries (e.g. trauma/fracture/surgery), increasing the rate of repair or recovery from chronic injuries or disease (e.g. inflammatory mediated diseases, osteoarthritis), and slowing or reducing the appearance or progression of diseases associated with aging (e.g. osteoporosis) in the skeletal system and other tissues.

A. Stromal-Derived Factor-1 (SDF-1)

It has been discovered that increasing the bioactivity of one or more active isoforms of SDF-1 increases autophagy and survival of stem cells, particularly mesenchymal stem cells. The chemokine stromal-derived factor-1 (SDF-1) was previously identified to regulates hematopoiesis, lymphocyte homing, B-lineage cell growth, and angiogenesis. It is constitutively expressed in most tissues as SDF-1α and SDF-1β resulting from alternative gene splicing. Other isoforms include: isoform SDF-1γ, which is mainly expressed in heart, with weak expression detected in several other tissues, and isoforms SDF-1δ, SDF-1ε and SDF-1θ which have highest expression levels in pancreas, with lower levels detected in heart, kidney, liver and spleen (Yu, et al., Gene, 374:174-179 (2006)).

1. Amino Acid Sequences of SDF-1

A consensus amino acid sequence for full-length SDF-1β is MNAKVVVVLV LVLTALCLSD GKPVSLSYRC PCRFFESHVA RANVKHLKIL NTPNCALQIV ARLKNNNRQV CIDPKLKWIQ EYLEKALNKR FKM

(SEQ ID NO:1), which includes a putative signal sequence of amino acids 1-21. SDF-1 is secreted.

A consensus sequence for full-length SDF-1α is amino acids 1-89 of SEQ ID NO:1.

Mature, secreted forms of SDF-1 are typically missing amino acids 1-21 of SEQ ID NO:1. Therefore, a consensus sequence for mature SDF-1β is

### KPVSLSYRCP CRFFESHVAR ANVKHLKILN TPNCALQIVA RLKNNNRQVC IDPKLKWIQE YLEKALNKRF KM

(SEQ ID NO:2), and a consensus sequence for mature SDF-1α is

### KPVSLSYRCP CRFFESHVAR ANVKHLKILN TPNCALQIVA RLKNNNRQVC IDPKLKWIQE YLEKALNK

### (SEQ ID NO:3).

Consensus sequences for the other isoforms for SDF-1 and variants of SDF-1 are known in the art, and can be identified with reference to SEQ ID NO:1, (see, for example, (UnitProtKB Accession No. P48061 (SDF1_HUMAN), version 137, which is specifically incorporated by reference herein in its entirety).

For example, full-length SDF-1γ (identifier: P48061-3; also known as: hSDF-1gamma; SDF-1 g) can have a consensus sequence

### MNAKVVVVLV LVLTALCLSD GKPVSLSYRC PCRFFESHVA RANVKHLKIL NTPNCALQIV ARLKNNNRQV CIDPKLKWIQ EYLEKALNKG RREEKVGKKE KIGKKKRQKK RKAAQKRKN

(SEQ ID NO:4); and mature SDF-1γ can have the sequence

### KPVSLSYRCP CRFFESHVAR ANVKHLKILN TPNCALQIVA RLKNNNRQVC IDPKLKWIQE YLEKALNKGR REEKVGKKEK IGKKKRQKKR KAAQKRKN

### (SEQ ID NO:5).

Full-length SDF-1δ (identifier: P48061-4; also known as: hSDF-1delta) can have a consensus sequence

### MNAKVVVVLV LVLTALCLSD GKPVSLSYRC PCRFFESHVA RANVKHLKIL NTPNCALQIV ARLKNNNRQV CIDPKLKWIQ EYLEKALNNL ISAAPAGKRV IAGARALHPS PPRACPTARA LCEIRLWPPP EWSWPSPGDV

(SEQ ID NO:6); and mature SDF-16 can have the sequence

### KPVSLSYRCP CRFFESHVAR ANVKHLKILN TPNCALQIVA RLKNNNRQVC IDPKLKWIQE YLEKALNNLI SAAPAGKRVI AGARALHPSP PRACPTARAL CEIRLWPPPE WSWPSPGDV

### (SEQ ID NO:7).

Full-length SDF-1ε (identifier: P48061-5; also known as: hSDFepsilon) can have a consensus sequence

### MNAKVVVVLV LVLTALCLSD GKPVSLSYRC PCRFFESHVA RANVKHLKIL NTPNCALQIV ARLKNNNRQV CIDPKLKWIQ EYLEKALNNC

(SEQ ID NO:8); and mature SDF-1ε can have the sequence

### KPVSLSYRCP CRFFESHVAR ANVKHLKILN TPNCALQIVA RLKNNNRQVC IDPKLKWIQE YLEKALNNC

### (SEQ ID NO:9).

Full-length SDF-1θ (identifier: P48061-6; also known as: hSDFphi; hSDFtheta; Phi) can have a consensus sequence

### MNAKVVVVLV LVLTALCLSD GKPVSLSYRC PCRFFESHVA RANVKHLKIL NTPNCALQIV ARLKNNNRQV CIDPKLKWIQ EYLEKALNKI WLYGNAETSR

(SEQ ID NO:10), and mature SDF-1θ can have the sequence

### KPVSLSYRCP CRFFESHVAR ANVKHLKILN TPNCALQIVA RLKNNNRQVC IDPKLKWIQE YLEKALNKIW LYGNAETSR

### (SEQ ID NO:11).

2. Functional Domains of SDF-1

Structure-function analysis of SDF-1α has identified the NH2-terminal amino acids (residues 1-8) as important for CXCR4 binding and activation (reviewed in De La Luz Sierra, et al., Blood, 103(7):2452-2459 (2004), which is specifically incorporated by reference herein in its entirety). Modification of the first 2 amino acids (K-1 and P-2) alone results in loss of receptor activation, and deletion of the first 8 amino acids results in loss of receptor binding activity.

However, the NH2-terminus alone, which is a highly mobile region of SDF-1α, was found to be insufficient for receptor binding and activation, and an additional site consisting of a RFFESH motif (residues 12-17 of SEQ ID NO:3) was identified as necessary for SDF-1α docking to CXCR4. Furthermore, the cluster of basic residues K-24, H-25, K-27, and R-41 (of SEQ ID NO:3) was proposed to provide surface charge complementarity for the negatively charged extracellular portion of CXCR4 and to contribute to a heparan sulfate binding site anchoring SDF-1α to cell surface proteoglycans.

Proteolytic degradation of endogenous SDF-1 in the bone marrow was identified as playing an important role in mobilization of hematopoietic progenitor cells to the peripheral circulation. Endogenous SDF-1 provides a retention signal for hematopoietic stem and progenitor cells, which express CXCR4 such that its local degradation would release the cells from this site. In vitro, SDF-1α can be enzymatically cleaved by metalloproteinases, CD26/dipeptidyl peptidase IV, serine proteases, and leukocyte elastase to generate distinct N-terminally truncated forms of the molecule.

Functional studies show that serum enzymes can selectively cleave SDF-1α both at the carboxy and NH2 terminus, and SDF-1β at the NH2 terminus only, generating molecules with differing specific activity. Processed forms SDF-1β (amino acids 3-72 of SEQ ID NO:2) and SDF-1α (amino acids 3-67 of SEQ ID NO:3) are produced after secretion by proteolytic cleavage of isoforms β and α, respectively. De La Luz Sierra, et al., Blood, 103(7):2452-2459 (2004) reported that when exposed to serum, SDF-1α (amino acids 1-68 of SEQ ID NO:3) undergoes processing first at the COOH terminus to produce SDF-1α (1-67) (amino acids 1-67 of SEQ ID NO:3) and then at the NH2 terminus to produce SDF-1α (3-67) (amino acids 3-67 of SEQ ID NO:3), whereas SDF-1β (1-72) (amino acids 1-72 of SEQ ID NO:2) is processed only at the NH2 terminus to produce SDF-1β (3-72) (amino acids 3-72 of SEQ ID NO:2).

The studies showed that proteolytic removal of the COOH-terminal K from SDF-1α reduced the polypeptide's ability to bind to heparin and to cells and to stimulate pre-B-cell proliferation and B-cell chemotaxis. The additional processing at the NH2 terminus reduces markedly SDF-1's ability to bind to heparin and to activate cells. The different sensitivity of SDF-1α and SDF-1β to proteolytic processing provides a mechanism for chemokine functional regulation and reveals a functional difference between the 2 splice forms of the chemokine.

3. Variants of SDF-1

Variants of SDF-1 and their functional activity are also known in the art. See, for example, UnitProtKB Accession No. P48061 (SDF1_HUMAN), version 137, last modified Jan. 9, 2013, which provides at least forty-two specific mutants with reference to SEQ ID NO:1, as well as their characterized function(s). Each of the mutants is hereby incorporated by reference for use in the compositions and methods provided herein as discussed in more detail below.

B. Compositions for Increasing Active or Agnostic Forms of SDF-1

1. SDF-1 Proteins and Polypeptides

Compositions for increasing the bioactivity of one or more active isoforms of SDF-1 can include one or more active isoforms of SDF-1. Active isoforms of SDF-1 include active forms of SDF-1α, SDF-1β, SDF-1γ, SDF-1δ, SDF-1ε, SDF-1θ, and active fragments and variants thereof. For example, in a preferred embodiment, a composition for increasing the bioactivity of SDF-1 includes a polypeptide the amino acid sequence of SEQ ID NO:1, 2, 3, 4, 5, 6, 7, 8, 10, or an active fragment or variant thereof. In some embodiments, the polypeptide includes the putative signal sequence (i.e., MNAKVVVVLVLVLTALCLSDG (SEQ ID NO:11), for example, SEQ ID NO:1, 4, 5, 6, 7, 8, 10, or an active fragment or variant thereof. In some embodiments, the amino acid sequence of SEQ ID NO:11 is substituted with an alternative signal sequence. n some embodiments, the polypeptide does not include the signal sequence. For example, the polypeptide can include the amino acid sequence of SEQ ID NO:2, 3, 5, 7, 9, or an active fragment or variant thereof.

In some embodiments, the active isoform of SDF-1 is an active fragment or variant of SDF-1α, SDF-1β, SDF-1γ, SDF-1δ, SDF-1ε, or SDF-1θ. The variant can have a sequence with 80%, 85%, 90%, 95%, 99%, or 100% sequence identity with SEQ ID NO:1, 2, 3, 4, 5, 6, 7, 8, 10, and preferably binds to and mediates signal transduction through CXCR4. Suitable active fragments and variants are known in the art and discussed in UnitProtKB Accession No. P48061 (SDF1_HUMAN), version 137. Preferably the polypeptide includes at least amino acids 1-17 of SEQ ID NO:2 or 3.

2. SDF-1 Fusion Proteins

Compositions for increasing the bioactivity of one or more active isoforms of SDF-1 can include a fusion protein including one or more active isoforms of SDF-1.

Fusion proteins containing one or more of the active SDF-1 polypeptides disclosed above can be coupled to other polypeptides to form fusion proteins. The presence of the second polypeptide can alter the solubility, stability, affinity and/or valency of the SDF-1 fusion polypeptide. As used herein, “valency” refers to the number of binding sites available per molecule. In one embodiment the second polypeptide is a polypeptide from a different source or different protein. Active SDF-1 fusion polypeptides have a first fusion partner comprising all or a part of an active SDF-1 protein fused (i) directly to a second polypeptide or, (ii) optionally, fused to a linker peptide sequence that is fused to the second polypeptide. The peptide/polypeptide linker domain can either be a separate domain, or alternatively can be contained within one of the other domains (SDF-1 polypeptide or second polypeptide) of the fusion protein.

SDF-1 exists as a monomer or homodimer; in equilibrium. Dimer formation is induced by non-acidic pH and the presence of multivalent anions, and by binding to CXCR4 or heparin. Monomeric form is required for full chemotactic activity and resistance to ischemia/reperfusion injury, whereas the dimeric form acts as a partial agonist of CXCR4, stimulating Ca2+ mobilization but with no chemotactic activity and instead acts as a selective antagonist that blocks chemotaxis induced by the monomeric form. Therefore, in some embodiments, the fusion protein optionally contains a domain that functions to dimerize or multimerize two or more fusion proteins. The domain that functions to dimerize or multimerize the fusion proteins can either be a separate domain, or alternatively can be contained within one of one of the other domains (SDF-1 polypeptide, second polypeptide or peptide/polypeptide linker domain) of the fusion protein. In one embodiment, the dimerization/multimerization domain and the peptide/polypeptide linker domain are the same.

Fusion proteins are of formula I:

N—R₁—R₂—R₃—C

wherein “N” represents the N-terminus of the fusion protein, “C” represents the C-terminus of the fusion protein, “R₁” is a SDF-1 polypeptide, “R₂” is an optional peptide/polypeptide linker domain, and “R₃” is a second polypeptide. Alternatively, R₃ may be the SDF-1 polypeptide and R₁ may be the second polypeptide.

The fusion proteins can be dimerized or multimerized. Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric.

a. Peptide or Polypeptide Linker Domain

The disclosed SDF-1 fusion proteins optionally contain a peptide or polypeptide linker domain that separates the SDF-1 polypeptide from the second polypeptide. Suitable peptide/polypeptide linker domains include naturally occurring or non-naturally occurring peptides or polypeptides. Peptide linker sequences are at least 2 amino acids in length. Preferably the peptide or polypeptide domains are flexible peptides or polypeptides. A “flexible linker” herein refers to a peptide or polypeptide containing two or more amino acid residues joined by peptide bond(s) that provides increased rotational freedom for two polypeptides linked thereby than the two linked polypeptides would have in the absence of the flexible linker. Such rotational freedom allows two or more antigen binding sites joined by the flexible linker to each access target antigen(s) more efficiently. Exemplary flexible peptides/polypeptides include, but are not limited to, the amino acid sequences Gly-Ser, Gly-Ser-Gly-Ser (SEQ ID NO:12), Ala-Ser, Gly-Gly-Gly-Ser (SEQ ID NO:13), (Gly₄-Ser)₃ (SEQ ID NO:14) and (Gly₄-Ser)₄ (SEQ ID NO:15).

Additional flexible peptide/polypeptide sequences are well known in the art. For example, the linker domain can contain the hinge region of an immunoglobulin. In a preferred embodiment, the hinge region is derived from a human immunoglobulin. Suitable human immunoglobulins that the hinge can be derived from include IgG, IgD and IgA. In a preferred embodiment, the hinge region is derived from human IgG. Amino acid sequences of immunoglobulin hinge regions and other domains are well known in the art.

b. SDF1-Ig Fusion Proteins

In some embodiments the active SDF-1 fusion protein is a SDF1-Ig fusion protein. In one embodiment, the second polypeptide contains one or more domains of an immunoglobulin heavy chain constant region, preferably having an amino acid sequence corresponding to the hinge, C_{H}2 and C_{H}3 regions of a human immunoglobulin Cγ1 chain or to the hinge, C_{H}2 and C_{H}3 regions of a murine immunoglobulin Cγ2a chain.

In a preferred dimeric fusion protein, the dimer results from the covalent bonding of Cys residue in the hinge region of two of the Ig heavy chains that are the same Cys residues that are disulfide linked in dimerized normal Ig heavy chains.

In one embodiment, the immunoglobulin constant domain may contain one or more amino acid insertions, deletions or substitutions that enhance binding to specific cell types, increase the bioavailability, or increase the stability of the SDF-1 fusion proteins, or fragments thereof. Suitable amino acid substitutions include conservative and non-conservative substitutions, as described above.

In another embodiment the second polypeptide may have a conjugation domain through which additional molecules can be bound to the SDF-1 fusion proteins. In one such embodiment, the conjugated molecule is capable of targeting the fusion protein to a particular organ or tissue. In another embodiment the conjugated molecule is Polyethylene Glycol (PEG).

The Fc portion of the fusion protein may be varied by isotype or subclass, may be a chimeric or hybrid, and/or may be modified, for example to improve effector functions, control of half-life, tissue accessibility, augment biophysical characteristics such as stability, and improve efficiency of production (and less costly). Many modifications useful in construction of disclosed fusion proteins and methods for making them are known in the art, see, for example, Mueller, et al., Mol. Immun., 34(6):441-452 (1997), Swann, et al., Cur. Opin. Immun., 20:493-499 (2008), and Presta, Cur. Opin. Immun. 20:460-470 (2008). In some embodiments the Fc region is the native IgG1, IgG2, or IgG4 Fc region. In some embodiments the Fc region is a hybrid, for example, a chimeric including IgG2/IgG4 Fc constant regions. Modifications to the Fc region include, but are not limited to, IgG4 modified to prevent binding to Fc gamma receptors and complement, IgG1 modified to improve binding to one or more Fc gamma receptors, IgG1 modified to minimize effector function (amino acid changes), IgG1 with altered/no glycan (typically by changing expression host), and IgG1 with altered pH-dependent binding to FcRn. The Fc region may include the entire hinge region, or less than the entire hinge region.

The therapeutic outcome in patients treated with rituximab (a chimeric mouse/human IgG1 monoclonal antibody against CD20) for non-Hodgkin's lymphoma or Waldenstrom's macroglobulinemia correlated with the individual's expression of allelic variants of Fcγ receptors with distinct intrinsic affinities for the Fc domain of human IgG1. In particular, patients with high affinity alleles of the low affinity activating Fc receptor CD16A (FcγRIIIA) showed higher response rates and, in the cases of non-Hodgkin's lymphoma, improved progression-free survival. In another embodiment, the Fc domain may contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA).

Another embodiment includes IgG2-4 hybrids and IgG4 mutants that have reduced binding to FcR which increase their half-life. Representative IG2-4 hybrids and IgG4 mutants are described in Angal, S. et al., Molecular Immunology, 30(1):105-108 (1993); Mueller, J. et al., Molecular Immunology, 34(6): 441-452 (1997); and U.S. Pat. No. 6,982,323 to Wang et al. In some embodiments the IgG1 and/or IgG2 domain is deleted for example, Angal et al. describe IgG1 and IgG2 having serine 241 replaced with a proline.

In a preferred embodiment, the Fc domain contains amino acid insertions, deletions or substitutions that enhance binding to CD16A. A large number of substitutions in the Fc domain of human IgG1 that increase binding to CD16A and reduce binding to CD32B are known in the art and are described in Stavenhagen, et al., Cancer Res., 57(18):8882-90 (2007). Exemplary variants of human IgG1 Fc domains with reduced binding to CD32B and/or increased binding to CD16A contain F243L, R929P, Y300L, V305I or P296L substitutions. These amino acid substitutions may be present in a human IgG1 Fc domain in any combination. In one embodiment, the human IgG1 Fc domain variant contains a F243L, R929P and Y300L substitution. In another embodiment, the human IgG1 Fc domain variant contains a F243L, R929P, Y300L, V305I and P296L substitution. In another embodiment, the human IgG1 Fc domain variant contains an N297Q substitution, as this mutation abolishes FcR binding.

3. SDF-1 Nucleic Acids

Isolated nucleic acid sequences encoding active SDF-1 polypeptides, fusions fragments and variants thereof are also disclosed herein. In some embodiments the nucleic acids are expressed in cells to produce the recombinant proteins discussed above. In some embodiments the nucleic acid molecules themselves are used in the composition. Therefore, in some embodiments, the composition for increasing the bioactivity of one or more active isoforms of SDF-1 includes a nucleic acid encoding one or more of the active SNF-1 polypeptides, or fragments, variants, or fusions thereof discussed above. The compositions can be used in ex vivo and in vivo methods of gene therapy to increase expression of an active form of SDF-1 in or around stem cells or stem cells niche(s).

a. Nucleic Acids Encoding SDF-1

As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that encode non-SDF-1 proteins). The term “isolated” as used herein with respect to nucleic acids also includes the combination with any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

The nucleic acid sequences encoding active SDF-1 polypeptides include genomic sequences. Also disclosed are mRNA sequence wherein the exons have been deleted. Other nucleic acid sequences encoding active SDF-1 polypeptides, such polypeptides that include the above-identified amino acid sequences and fragments, variants, fusions thereof, are also disclosed. Nucleic acids encoding active SDF-1 polypeptides, for fragments, variants or fusions thereof may be optimized for expression in the expression host of choice. Codons may be substituted with alternative codons encoding the same amino acid to account for differences in codon usage between the organism from which the SDF-1 nucleic acid sequence is derived and the expression host. In this manner, the nucleic acids may be synthesized using expression host-preferred codons.

Nucleic acids can be in sense or antisense orientation, or can be complementary to a reference sequence encoding a SDF-1 polypeptide. Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev., 7:187-195; and Hyrup et al. (1996) Bioorgan. Med. Chem., 4:5-23. In addition, the deoxyphosphate backbone can be replaced with, for example, a phosphorothioate or phosphorodithioate backbone, a phosphoroamidite, or an alkyl phosphotriester backbone.

Nucleic acids encoding polypeptides can be administered to subjects in need thereof. Nucleic delivery involves introduction of “foreign” nucleic acids into a cell and ultimately, into a live animal. Compositions and methods for delivering nucleic acids to a subject are known in the art (see Understanding Gene Therapy, Lemoine, N. R., ed., BIOS Scientific Publishers, Oxford, 2008).

b. Vectors and Host Cells

Vectors encoding SDF-1 polypeptides, and fragments, variants and fusions thereof are also provided. Nucleic acids, such as those described above, can be inserted into vectors for expression in cells. The vectors can be used for production of recombinant protein, or in methods of gene therapy. As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. For example, the control sequence can be incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

An expression vector can include a tag sequence. Tag sequences are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, Conn.), maltose E binding protein and protein A. In one embodiment, a nucleic acid molecule encoding a SDF-1 fusion polypeptide is present in a vector containing nucleic acids that encode one or more domains of an Ig heavy chain constant region, preferably having an amino acid sequence corresponding to the hinge, C_{H}2 and C_{H}3 regions of a human immunoglobulin Cγ1 chain.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the SDF-1 polypeptides or fusion polypeptides described herein.

The vectors can be used to express SDF-1 in cells. An exemplary vector includes, but is not limited to, an adenoviral vector. One approach includes nucleic acid transfer into primary cells in culture followed by autologous transplantation of the ex vivo transformed cells into the host, either systemically or into a particular organ or tissue. Ex vivo methods can include, for example, the steps of harvesting cells from a subject, culturing the cells, transducing them with an expression vector, and maintaining the cells under conditions suitable for expression of the encoded polypeptides. These methods are known in the art of molecular biology. The transduction step can be accomplished by any standard means used for ex vivo gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject. In one embodiment, expression vectors containing nucleic acids encoding fusion proteins are transfected into cells that are administered to a subject in need thereof.

In vivo nucleic acid therapy can be accomplished by direct transfer of a functionally active DNA into mammalian somatic tissue or organ in vivo. Nucleic acids may also be administered in vivo by viral means. Nucleic acid molecules encoding polypeptides or fusion proteins may be packaged into retrovirus vectors using packaging cell lines that produce replication-defective retroviruses, as is well-known in the art. Other virus vectors may also be used, including recombinant adenoviruses and vaccinia virus, which can be rendered non-replicating. In addition to naked DNA or RNA, or viral vectors, engineered bacteria may be used as vectors.

Nucleic acids may also be delivered by other carriers, including liposomes, polymeric micro- and nanoparticles and polycations such as asialoglycoprotein/polylysine.

In addition to virus- and carrier-mediated gene transfer in vivo, physical means well-known in the art can be used for direct transfer of DNA, including administration of plasmid DNA and particle-bombardment mediated gene transfer.

4. Other Compounds that Increase the Bioactivity of SDF-1

a. Small Molecules

In some embodiments the factor that increases bioavailability of an active isoform of SDF-1 is a small molecule.

An exemplary small molecule is metformin, or a pharmacologically active salt, thereof. Metformin is a biguanide that is mainly known for its antihyperglycaemic activity and is widely used in the treatment of non-insulin dependent diabetes. The Examples below show that metformin also increase expression of endogenous SDF-1.

When preparing the pharmaceutical formulations of the invention, metformin can be used either as the free base or in the form of a pharmaceutically acceptable acid addition salt thereof such as the hydrochloride, acetate, benzoate, citrate, fumarate, embonate, chlorophenoxyacetate, glycolate, palmoate, aspartate, methanesulphonate, maleate, parachlorophenoxyisobutyrate, formate, lactate, succinate, sulphate, tartrate, cyclohexanecarboxylate, hexanoate, octonoate, decanoate, hexadecanoate, octodecanoate, benzenesulphonate, trimethoxybenzoate, paratoluenesulphonate, adamantanecarboxylate, glycoxylate, glutamate, pyrrolidonecarboxylate, naphthalenesulphonate, 1-glucosephosphate, nitrate, sulphite, dithionate or phosphate.

Metformin can be administered in an immediate release or slow release formulation. In another embodiment, the composition can be a slow release formulation with, for example, reduced amount of active substance. Different sustained release formulations of metformin are described in patents such as U.S. Pat. No. 6,475,521, U.S. Pat. No. 5,972,389, EP patent no. 1,335,708.

The amount of metformin or one of its pharmaceutically acceptable salts can be from about 100 mg to 2000 mg, preferably from 200 mg to 1500 mg, more preferably from about 500 mg to 1000 mg.

Dosages and formulations of metformin are well known in the art. For example, metformin can be administered in the form of a tablet for once-a-day administration, twice-a-day, three times a day, or four times a day.

In some embodiments, the metformin is administered in a dosage escalation protocol. According to the Physician's Desk Reference, for treatment for Type II diabetes the starting dose should be 500 mg of metformin twice a day; or 850 mg of metformin once a day. After one week, the dose of metformin can be increased to 1000 mg as the first dose of the day and 500 mg as the second dose. After another week, the dose can be increased to 1000 mg of metformin two times a day. The maximum safe dose described in the Physician's Desk Reference is 2550 mg a day (which should be taken as 850 mg three times a day).

Also according to the Physician's Desk Reference, clinically significant responses in Type II diabetics are not seen at doses below 1500 mg a day of metformin, however, dosages as low as 500 mg twice a day have been proscribed to healthy non-diabetics who are seeking to obtain metformin's other proven benefits such as enhancing insulin sensitivity and reducing excess levels of insulin, glucose, cholesterol and triglycerides in the blood (LifeExtension, Metformin Dosage, 2013). The preferred dosage for each subject can be determined by one of skill in the art. For example, some individuals may benefit from 500 mg twice a day, while others may need 1000 mg twice a day for optimal effects.

Metformin is sold under several trade names, including GLUCOPHAGE XR, CARBOPHAGE SR, RIOMET, FORTAMET, GLUMETZA, OBIMET, GLUFORMIN, DIANBEN, DIABEX, AND DIAFORMIN.

Metformin IR (immediate release) is available in 500 mg, 850 mg, and 1000 mg tablets. Metformin SR (slow release) or XR (extended release) was introduced in 2004, in 500 mg and 750 mg strengths, mainly to counteract the most common gastrointestinal side effects, as well as to increase compliance by reducing pill burden. No difference in effectiveness exists between the two preparations.

Metformin is freely soluble in water. It is also known to be a poorly compressible substance. A poorly compressible substance is one that does not bind to form a tablet upon application of compression force. Therefore, such substances may require additional processing and special formulating before they can be compressed into tablets. With such substances, the additional processing necessary is usually a wet granulation step, as direct compression would not be effective. These substances may be formulated with binders or other materials that have high binding capacity (or that act as an aid to compressibility) such that the non-bonding properties of the non-compressible material are overcome. Other techniques to assist compression include having residual moisture in the blend prior to compression or having the non-compressible material in very low amounts in the tablet formulation. High-dose drugs, such as metformin, do not lend themselves to direct compression because of the relatively low proportion of diluent or compression aid in the tablet, poor powder flow and poor compressibility.

b. Transcription Factors

In some embodiments, the composition includes a compound that increases bioactivity of one or more active isoforms of SDF-1 by increasing expression of one or more active isoforms of SDF-1. Such compounds include one or more factors that increase the expression of or increase the half-life of endogenous SDF-1, preferably SDF-1β. Factors that increase expression of endogenous SDF-1 include, for example, SDF-1 transcription factors. SDF-1 transcription factors can be provided as a recombinant polypeptide, or an isolated nucleic acid encoding the transcription factor for example in the form of an expression vector or transfectable mRNA.

c. Inhibitors or Antagonists of SDF-1 miRNA

The agent can be an antagonist of an miRNA that inhibits expression of one or more isoforms of SDF-1. The antagonist can be, for example, a small molecule, a polypeptide such as a nuclease, or a functional nucleic acid.

Therefore, in some embodiments the agent is a functional nucleic acid designed to reduce or inhibit the expression or activity of an miRNA that targets one or more isoforms SDF-1. For example, antisense oligonucleotides, RNAi, dsRNA, miRNA, siRNA, external guide sequences, and the like can be designed to target miRs such as 29a-5p, 1244, 141, 144, 200a, 200c, or a combination thereof, or variants thereof having 70%, 75%, 80%, 85%, 90%, 95%, 97%, 99% or more sequence identity to miRs 29a-5p, 1244, 141, 144, 200a, or 200c.

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include, but are not limited to, antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10-12 M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a K_{d} with the target molecule at least 10, 100, 1000, 10,000, or 100,000 fold lower than the IQ with a background binding molecule. It is preferred when doing the comparison for a polypeptide for example, that the background molecule be a different polypeptide.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukarotic cells. Representative examples of how to make and use EGS molecules to facilitate cleavage of a variety of different target molecules are known in the art.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett., 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for the herein disclosed transferases.

In some embodiments the functional nucleic acid is encoded by an expression vector that is transfected into cells that expression the target gene or mRNA.

C. Compositions for Decreasing Inactive or Antagonistic Forms SDF-1

Compositions for increasing the bioactivity of one or more active isoforms of SDF-1 can include an agent that decreases or reduces the level or production of inactive or antagonistic forms of SDF-1. Inactive and antagonistic forms of SDF-1 include forms of SDF-1, or variants, fragments, or fusion there of that cannot be bind to CXCR4 (i.e., inactive forms), or that bind to CXCR4 but cannot mediate CXCR4 signal transduction (i.e., antagonist forms). Exemplary inactive and antagonistic forms of SDF-1 are discussed above and include, for example, SEQ ID NO:2 or 3 without N-terminal amino acids 1-8; SEQ ID NO:2 or 3 without amino acids 12-17, or a polypeptide consisting of amino acids 3-72 of SEQ ID NO:2, or amino acids 3-67 of SEQ ID NO:3. Endogenous inactive and antagonistic forms of SDF-1 can be produce by proteolytic degradation.

For example, it is known in the art that active isoforms of SDF-1 can be processed to inactive or antagonistic forms when enzymatically cleaved by metalloproteinases, CD26/dipeptidyl peptidase IV, serine proteases, and leukocyte elastase to generate distinct N-terminally truncated forms of the molecule. Therefore the agent can be an agent that inhibits or reduces the expression or activity of a protease that degrades active form of SDF-1, such as a metalloproteinase, CD26/dipeptidyl peptidase IV, serine proteases, and leukocyte elastase.

1. Small Molecules

An agent that decreases or reduces the level or production of inactive or antagonistic forms of SDF-1 can be a small molecule that reduces or inhibits the expression of or activity of a protease that degrades active forms of SDF-1, such as a metalloproteinase, CD26/dipeptidyl peptidase IV, serine proteases, and leukocyte elastase.

The agent can be, for example, a small molecule that reduces or inhibits the activity of dipeptidyl peptidase IV (DPP4). DPP4, also known as adenosine deaminase complexing protein 2 or CD26 (cluster of differentiation 26) is a protein that, in humans, is encoded by the DPP4 gene. DPP4 is an intrinsic membrane glycoprotein enzyme expressed on the surface of most cell types and is associated with immune regulation, signal transduction and apoptosis. It is a serine exopeptidase that cleaves X-proline dipeptides from the N-terminus of polypeptides.

DPP4 inhibitors are known in the art and typically characterized as oral hypoglycemics used to treat Type II diabetes mellitus because they reduce glucagon and blood glucose levels. The mechanism of DPP-4 inhibitors diabetes treatment is to increase incretin levels (GLP-1 and GIP), which inhibit glucagon release, which in turn increases insulin secretion, decreases gastric emptying, and decreases blood glucose levels. DDP4 inhibitors include, but are not limited to, sitagliptin (FDA approved 2006, marketed by Merck & Co. as JANUVIA), vildagliptin (EU approved 2007, marketed in the EU by Novartis as GALVUS), saxagliptin (FDA approved in 2009, marketed as ONGLYZA), linagliptin (FDA approved in 2011, marketed as TRAJENTA by Eli Lilly Co and Boehringer Ingelheim), dutogliptin (being developed by Phenomix Corporation), gemigliptin (being developed by LG Life Sciences, Korea), alogliptin (FDA approved 2013, marketed by Takeda Pharmaceutical Company), and pharmaceutically acceptable salts, and active analogs thereof.

Dosages for specific DDP4 inhibitors are known in the art and can be adjusted by one of skill in the art according to the specific subject and indication to be treated. Generally DPP4 inhibitors are administered in dosages of between about 1 mg and 1000 mg.

For example, a dosage for sitagliptin can be between about 2.5 mg and 100 mg once a day. For example, a dosage can be about 50 mg per day, or 100 mg per day. Exemplary dosages are provided below.

A dosage for vildagliptin is typically between about 25 mg and 200 mg. For example, a dosage can be about 50 mg per day, or 100 mg per day.

A dosage for saxagliptin can be between about 2.5 mg and 400 mg per day. For example, a dosage can be about 2.5 mg and 5 mg once a day.

A dosage for linagliptin can be between about 1 mg and 10 mg. For example, in some embodiments, the dosage is 5 mg once a day.

A dosage for dutogliptin can be between about 25 mg and 800 mg. For example, in some embodiments, the dosage is 400 mg or 200 mg per day.

A dosage for gemigliptin can be between about 25 mg and 500 mg. For example, in some embodiments, the dosage is 50 mg, 100 mg or 200 mg per day.

A dosage for alogliptin can be between about 10 mg and 500 mg. For example, in some embodiments, the dosage is 12.5 mg, 25 mg, 100 mg or 400 mg per day.

2. Gene Therapy

An agent that decreases or reduces the level or production of inactive or antagonistic forms of SDF-1 can be a functional nucleic acid that reduces or inhibits the expression or activity of a protease that degrades active forms of SDF-1, such as a metalloproteinase, CD26/dipeptidyl peptidase IV, serine proteases, and leukocyte elastase.

Therefore, in some embodiments the agent is a functional nucleic acid designed to reduce or inhibit the expression or activity of a nucleic acid, such as a gene or mRNA, encoding a metalloproteinase, CD26/dipeptidyl peptidase IV, serine proteases, and leukocyte elastase. For example, the composition can include a functional nucleic acid designed to reduce or inhibit expression or activity of DPP4. Genomic and mRNA/cDNA sequences of DPP4 are known in the art, see for example NCBI Reference Sequence: NC_{—}000002.11, Homo sapiens chromosome 2, GRCh37.p10 Primary Assembly; and NCBI Reference Sequence: NM_{—}001935.3, Homo sapiens dipeptidyl-peptidase 4 (DPP4), mRNA, each of which is specifically incorporated by reference in its entirety, and can be used to designed functional nucleic acids that target DPP4.

In some embodiments, the functional nucleic acid is a miRNA that targets expression of DPP4. Examples of miRNA that target DPP4 are known in the art and include miR-3173-5p.

In some embodiments the functional nucleic acid is encoded by an expression vector that is transfected into cells that expression the target gene or mRNA.

D. Compositions for Increasing the Bioactivity of Receptors of SDF-1

The composition can include an agent that increases expression of one or more receptors of SDF-1. The agent can be, for example, a nucleic acid encoding the receptor. Receptors for SDF-1 are known in the art and include CXCR4 and CXCR7. Nucleic acid sequences for CXCR4 and CXCR7 are known in the art. See, for example, NCBI Reference Sequence: NM_{—}001008540.1, Homo sapiens chemokine (C-X-C motif) receptor 4 (CXCR4), transcript variant 1, mRNA; and NCBI Reference Sequence: NM_{—}020311.2: Homo sapiens chemokine (C-X-C motif) receptor 7 (CXCR7), mRNA, each of which is specifically incorporated by reference herein in its entirety.

The compositions can include an isolated nucleic acid encoding SDF-1 receptor, for example in the form of an expression vector, transfectable mRNA, or other form suitable for increasing cell-surface expression of the receptor or receptors in a cell, such as a stem cell. Isolated nucleic acids and vectors for expressing constructs in cells are discussed above with respect to compositions for increasing the level of active isoforms of SDF-1.

The agent can also be a functional nucleic acid that targets an miRNA that reduces or inhibits expression of an endogenously expressed CXCR4 or CXCR7. miRNAs that target receptors of SDF-1 are known in the art. For example, miR-3120-3p is an miRNA that targets CXCR4. Therefore, in some embodiments, the composition for increasing expression of CXC4 includes a functional nucleic acid that reduces or inhibits the expression or activity of miR-3120-3p. Functional nucleic acids are discussed above with respect to compositions for reducing inhibitors or antagonists of SDF-1 mRNA.

S1P receptors, b-arrestin, G-CSF, and GM-CSF are believed to regulate the expression or activity of SDF-1 receptors. Therefore, in some embodiments, the expression or activity of S1P receptors, b-arrestin, G-CSF, and GM-CSF are manipulated to increase expression, activity or bioavailability of SDF-1 receptors.

E. Pharmaceutical Compositions

Pharmaceutical compositions including the polypeptides, fusion proteins, nucleic acids, and small molecules are provided. Pharmaceutical compositions can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some in vivo approaches, the compositions are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the polypeptides, fusion proteins, nucleic acids, small molecules, or combinations thereof, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 10 mg/kg of body weight daily are administered to mammals. Generally, for intravenous injection or infusion, dosage may be lower.

In certain embodiments, the compositions are administered locally, for example by injection directly into a site to be treated. Typically, local injection causes an increased localized concentration of the compositions which is greater than that which can be achieved by systemic administration.

1. Formulations for Parenteral Administration

The compositions disclosed herein, including those containing peptides and polypeptides, can be administered in an aqueous solution, by parenteral injection. The formulation may also be in the form of a suspension or emulsion. In general, pharmaceutical compositions are provided including effective amounts of a polypeptide, fusion protein, nucleic acid, small molecule, or combinations thereof and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Formulations for Topical Administration

The polypeptides, fusion proteins, nucleic acids, small molecules, or combinations thereof can be applied topically. Topical administration can include application to the lungs, nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent® nebulizer (Mallinckrodt Inc., St. Louis, Mo.); the Acorn® II nebulizer (Marquest Medical Products, Englewood, Colo.); the Ventolin® metered dose inhaler (Glaxo Inc., Research Triangle Park, N.C.); and the Spinhaler® powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

Transdermal formulations may also be prepared. These will typically be ointments, lotions, sprays, or patches, all of which can be prepared using standard technology. Transdermal formulations can include penetration enhancers.

3. Implants, Coatings, and Sustained or Controlled Delivery Polymeric Matrices

The polypeptides, fusion proteins, nucleic acids, small molecules, and combinations thereof can be administered in sustained or other controlled release formulations. Controlled release polymeric devices can be made for long term release systemically following implantation of a polymeric device (rod, cylinder, film, disk) or injection (microparticles). The matrix can be in the form of microparticles such as microspheres, where the polypeptide, fusion protein, nucleic acid, small molecule, or combinations thereof are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer can be cast as a thin slab or film, ranging from nanometers to four centimeters, a powder produced by grinding or other standard techniques, or even a gel such as a hydrogel.

Either non-biodegradable or biodegradable matrices can be used for delivery of polypeptides, fusion proteins, nucleic acids, small molecules, or combinations thereof, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

In another embodiment, the polypeptides, fusion proteins, nucleic acids, small molecules, or combinations thereof are administered with transplanted cells encapsulated within a matrix to allow release of the composition over a period of time in the area of transplantation. The matrix can be a polymeric matrix made using any polymer suitable for cell encapsulation. Exemplary polymeric materials suitable for encapsulating cells include, but are not limited to, alginate, agarose, hyaluronic acid, collagen, synthetic monomers, albumin, fibrinogen, fibronectin, vitronectin, laminin, dextran, dextran sulfate, chondroitin sulfate, dermatan sulfate, keratan sulfate, chitin, chitosan, heparan, heparan sulfate, or a combination thereof.

F. Combination Therapies

In some embodiments, two or more agents for increasing the bioavailability of an active isoform of SDF-1 are co-administered. The agents can be administered in the same pharmaceutical composition or separate pharmaceutical compositions.

In some embodiments, the agent or agents for increasing the bioavailability of an active isoform of SDF-1 are administered in combination with second therapeutic agent that does not increase the bioavailability of an active isoform of SDF-1. The second therapeutic agent can be an agent that increases autophagy, reduces apoptosis, or increases survival of cells, particularly stem cells. In some embodiments the second therapeutic agent increases autophagy induction mediated by non-SDF-1/axis mechanisms, for example, by induction of mTOR regulated pathways. An example of an agent that regulates mTOR pathways is rapamycin.

In some embodiments, the agent or agents for increasing the bioactivity of one or more active isoforms of SDF-1 is a conventional therapeutic agent used for treatment of the disease or condition being treated. Conventional therapeutics agents are known in the art and can be determined by one of skill in the art based on the disease or disorder to be treated. For example, if the disease or condition is enhancing the survival of a transplant, the agent for increasing one or more active isoforms of SDF-1 may be co-administered with an immunosuppressant.

### III. Methods of Manufacture

A. Methods for Producing Polypeptides

Isolated polypeptides can be obtained by, for example, chemical synthesis or by recombinant production in a host cell. To recombinantly produce a polypeptide, including a fusion protein, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding the fusion protein. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well known in the art include, for example, Escherichia coli strains such as BL-21, and cultured mammalian cells such as CHO cells.

In eukaryotic host cells, a number of viral-based expression systems can be utilized to express polypeptides. Viral based expression systems are well known in the art and include, but are not limited to, baculoviral, SV40, retroviral, or vaccinia based viral vectors.

Mammalian cell lines that stably express variant polypeptides can be produced using expression vectors with appropriate control elements and a selectable marker. For example, the eukaryotic expression vectors can be used to express polypeptides in Chinese hamster ovary (CHO) cells, COS-1 cells, human embryonic kidney 293 cells, NIH3T3 cells, BHK21 cells, MDCK cells, and human vascular endothelial cells (HUVEC). Additional suitable expression systems include the GS Gene Expression System™ available through Lonza Group Ltd.

Following introduction of an expression vector by electroporation, lipofection, calcium phosphate, or calcium chloride co-precipitation, DEAE dextran, or other suitable transfection method, stable cell lines can be selected (e.g., by metabolic selection, or antibiotic resistance to G418, kanamycin, or hygromycin or by metabolic selection using the Glutamine Synthetase-NS0 system). The transfected cells can be cultured such that the polypeptide of interest is expressed, and the polypeptide can be recovered from, for example, the cell culture supernatant or from lysed cells. Alternatively, a fusion protein can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Polypeptides can be isolated using, for example, chromatographic methods such as affinity chromatography, ion exchange chromatography, hydrophobic interaction chromatography, DEAE ion exchange, gel filtration, and hydroxylapatite chromatography. In some embodiments, polypeptides can be engineered to contain an additional domain containing amino acid sequence that allows the polypeptides to be captured onto an affinity matrix. For example, an Fc-fusion polypeptide in a cell culture supernatant or a cytoplasmic extract can be isolated using a protein A column. In addition, a tag such as c-myc, hemagglutinin, polyhistidine, or Flag™ (Kodak) can be used to aid polypeptide purification. Polypeptide enhancing amino acid sequence such as SUMO/SMT3 can also be added to increase expression of the polypeptide of interest. Such tags can be inserted anywhere within the polypeptide, including at either the carboxyl or amino terminus. In some embodiments, the tag is following expression of the polypeptide. Other fusions that can be useful include enzymes that aid in the detection of the polypeptide, such as alkaline phosphatase. Immunoaffinity chromatography also can be used to purify polypeptides. Polypeptides can additionally be engineered to contain a secretory signal (if there is not a secretory signal already present) that causes the polypeptide to be secreted by the cells in which it is produced. The secreted polypeptide can then be isolated from the cell media.

B. Methods for Producing Isolated Nucleic Acid Molecules

Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid encoding a polypeptide or inhibitory nucleic acid. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA, 87:1874-1878; and Weiss (1991) Science, 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Polypeptide or inhibitory nucleic acid encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of amino acid positions that can be modified include those described herein.

### IV. Methods of Use

It has been discovered that autophagy is an important factor in stem cell survival. As discussed in the Examples below, it has been discovered that the SDF-1 (CXCL12) axis can mediate mesenchymal stem cell (MSC) survival by enhancing the autophagic cell pro-survival pathways, and by reducing apoptotic cell death pathways. Accordingly, methods of increasing autophagy in cells, particularly stem cells, are disclosed. The methods typically include contacting a cell or population of cells with an effective amount of an agent that increases the bioactivity of an active isoform of SDF-1 to increase autophagy of the cell or cells. The contacting can occur in vivo or in vitro. The increase in autophagy can be compared to a control, for example cells that are not contacted with the agent.

An increase in autophagy can be measured using methods that are known in the art. For example, increased autophagy is associated with increased viability and survival of cells over time. In some embodiments, the composition is administered in an effective amount to increase viability or survival of a cell or population of cells compared to a control.

Markers of autophagy and autophagy-related pathways are known in the art, and discussed in the Examples below. The induction of autophagy utilizes two ubiquitin-like conjugation systems as part of the vesicle elongation process. One pathway involves the covalent conjugation of Atg12 to Atg5 and the second pathway comprises the conjugation of phosphatidylethanolamine to LC3/Atg8. Lipid conjugation then leads to the conversion of the soluble form of LC3-I to the autophagosome-associated form LC3-II (Tanida, et al., Int J Biochem Cell Biol, 36:2503-2518 (2004)). Another key player involved in the onset of autophagy is beclin 1. Like other BH3-only proteins, beclin 1 interacts with anti-apoptotic multi-domain proteins of the Bcl-2 family via its BH3 domain, and this interaction can be competitively disrupted to liberate beclin 1 and stimulate autophagy (Maiuri, et al., EMBO J., 26:2527-2539 (2007); Kessel, et al., Cancer Lett., 249:294-299 (2007); Daido, et al., Cancer Res., 64:4286-4293 (2004); Hamacher-Brady, et al., Cell Death Differ., 14:146-157 (2007); Oberstein, et al., J. Biol. Chem., 282:13123-13132 (2007); Maiuri, et al., Autophagy, 3:374-376 (2007); Nobukuni, et al., Curr. Opin. Cell Biol., 19:135-141 (2007)). Therefore, in some embodiments, the composition is administered in an effective amount to increase the expression or activity of one or more markers associated with autophagy or an autophagy associated pathway. Markers include, but are not limited to, LC3-II and beclin 1.

Morphological markers of autophagy are also known in the art. Three forms of autophagy have been described, which in general mediate highly regulated mechanisms of cell survival. Macroautophagy (hereafter referred to as autophagy) involves the bulk turnover of cytoplasmic proteins, including damaged or pathologically aggregated proteins, in a generalized fashion as part of a constitutive homeostatic recycling process. Autophagy can be increased in response to stress to provide needed nutrients and energy for cellular survival; however, when extreme levels of autophagy are induced, it can also lead to “autophagic cell death” (Shintani, Science, 306:990-995 (2004); Rubensztein, et al., Nat. Rev. Drug Discov., 6:304-312 (2007), Kroemer, et al., Nat. Rev. Mol. Cell Biol., 9:1004-1010 (2008)). Furthermore, autophagy can also specifically target distinct organelles (e.g., mitochondria in mitophagy or the endoplasmic reticulum (ER) in reticulophagy), thereby eliminating supernumerary or damaged cell structures (Shintani, Science, 306:990-995 (2004); Rubensztein, et al., Nat Rev Drug Discov, 6:304-312 (2007), Kroemer, et al., Nat Rev Mol Cell Biol, 9:1004-1010 (2008)). During autophagy, parts of the cytoplasm and intracellular organelles are sequestered within characteristic double- or multi-membrane autophagosomes and eventually delivered to lysosomes for bulk degradation (Shintani, Science, 306:990-995 (2004); Rubensztein, et al., Nat Rev Drug Discov, 6:304-312 (2007), Kroemer, et al., Nat Rev Mol Cell Biol, 9:1004-1010 (2008)). The compositions disclosed herein can be useful for increasing macrophagy, mitophagy, reticulophagy, or combinations thereof.

In some embodiments, the increase in autophagy is measured by examining morphological markers of autophagy, for example, increased formation of autophagosomes or changes to the morphology or number of mitochondria or lysosomes.

Other methods of detecting autophagy are described in Munaflo ad Colombo et al., J. Cell Science, 114:3619-3629 (2001), and commercially available assays are available through Clontech (i.e., pAutophagSENSE), Invitrogen (i.e., PREMO™ Autophagy Sensor, LC3B Antibody Kit, PREMO™ Autophagy TR-FRET Assay), and SABiosciences (i.e., Autophagy PCR Array). Reagents for visualizing organelles in cells are also commercially available, see, for example, MITOTRACKER®, LYSOTRACKER®, and ORGANELLE LIGHTS™ (Life Technologies).

In some embodiments, the methods of increasing autophagy also reduce apoptosis. Apoptosis is a set of well-described forms of programmed cell death, which involves the activation of proteolytic enzymes in signaling cascades leading to the rapid destruction of cellular organelles and chromatin (Danial, et al., Cell, 116:205-219 (2004); Green, Cell, 121:671-674 (2005). It has been discovered SNF-1-mediated increases in autophagy can coincide with a decrease in pro-apoptotic factors. For example, a decrease in levels of cleaved caspase-3, resulting in decreased levels of cleaved PARP and, in turn, increased levels of intact PARP relative to controls. Therefore, the composition can be administered in an effective amount to decrease expression of pro-apoptotic factors in a cell. In some embodiments, the composition is administered in an effective amount to reduce levels of cleaved caspase-3, to decrease levels of cleaved PARP, to increase levels of intact PARP, or combinations thereof compared to a control. Other markers of apoptosis are known in the art and can be used to measure differences in apoptosis between cells treated with a composition that increases the bioactivity of an isoform SDF-1 and a control.

A. Therapeutic Strategies

1. Cell Types

The compositions and methods described herein can be used to increase autophagy in cells in vitro, ex vivo, or in vitro. Suitable target cells typically express one or more receptors for an active isoform of SDF-1, such as CXCR4, CXCR7, or a combination thereof, and can include, but are not limited to, primary cells and established cell lines, embryonic cells, immune cells, stem cells, and differentiated cells including, but not limited to, cells derived from ectoderm, endoderm, and mesoderm, including fibroblasts, parenchymal cells, hematopoietic cells, and epithelial cells.

Cells can include progenitor cells, unipotent cells, multipotent cells, and pluripotent cells; embryonic stem cells, inner mass cells, bone marrow cells, cells from umbilical cord blood. The cells can be ectoderm, mesoderm, or endoderm, or cells derived therefrom. The cells can be adult stem cells such as hematopoietic stem cells, mesenchymal stem cells, epithelial stem cells, and muscle satellite cells. In some embodiments the cells are induce pluripotent stem (iPS) cells. In a preferred embodiment, the cells are stem cells.

The cells can be stem cells, or progenitor cells, from various adult tissues including, but not limited to, neuronal/brain, cardiac muscle, skeletal muscle, gastrointestinal, skin, liver, kidney, adipose, etc.).

In a preferred embodiment the cells are mesenchymal stem cells. Mesenchymal stem cells, or MSCs, are multipotent stromal cells that can differentiate into a variety of cell types, including: osteoblasts (bone cells), chondrocytes (cartilage cells), and adipocytes (fat cells). The MSCs can be isolated from bone marrow.

In some embodiments, the cells are bone marrow stem cells, such as mesenchymal stem cells, or stem cells from cardiac tissue.

2. In Vitro and Ex Vivo Methods

The compositions and methods described herein can be used to increase autophagy in cells in vitro or ex vivo. The method typically involves contacting the cells with an effective amount of an agent that increases the bioavailability of one or more active isoforms of SDF-1 to increase autophagy in the cell. In some embodiments the composition is administered in an effective amount to increase survival or decrease in the cell, or a population of cells. The cells can primary cells isolated from a subject, or cells of an established cell line. The cells can be of a homogenous cell type, or can be a heterogeneous mixture of different cells types. For example, the cells can be from a heterogenous cell line possessing cells of different types, such as in a feeder cell culture, or a mixed culture in various states of differentiation. The cells can be a transformed cell line that can be maintained indefinitely in cell culture.

Any eukaryotic cell can contacted with the compositions to increase autophagy of the cell. In a preferred embodiment, the cells are stem cells. Preferably the cells express a receptor for SDF-1, for example CXCR4, CXCR7, or a combination thereof.

The methods are particularly useful in the field of personalized therapy, for example, to prepare cells for transplant or engraftment. For example, target cells are first isolated from a donor using methods known in the art, contacted with the composition for increasing autophagy in vitro (ex vivo), and administered to a patient in need thereof. Sources or cells include cells harvested directly from the patient or an allographic donor. In preferred embodiments, the cells to be administered to a subject will be autologous, e.g. derived from the subject, or syngenic. Allogeneic cells can also be isolated from antigenically matched, genetically unrelated donors (identified through a national registry), or by using target cells obtained or derived from a genetically related sibling or parent.

Cells can be selected by positive and/or negative selection techniques. For example, antibodies binding a particular cell surface protein may be conjugated to magnetic beads and immunogenic procedures utilized to recover the desired cell type. It may be desirable to enrich the target cells prior to transient transfection. As used herein in the context of compositions enriched for a particular target cell, “enriched” indicates a proportion of a desirable element (e.g. the target cell) which is higher than that found in the natural source of the cells. A composition of cells may be enriched over a natural source of the cells by at least one order of magnitude, preferably two or three orders, and more preferably 10, 100, 200, or 1000 orders of magnitude. Once target cells have been isolated, they may be propagated by growing in suitable medium according to established methods known in the art. Established cell lines may also be useful in for the methods. The cells can be stored frozen if necessary.

The cells can be encapsulated within a matrix, such as a polymeric matrix, using suitable polymers, including, but not limited to alginate, agarose, hyaluronic acid, collagen, synthetic monomers, albumin, fibrinogen, fibronectin, vitronectin, laminin, dextran, dextran sulfate, chondroitin sulfate, dermatan sulfate, keratan sulfate, chitin, chitosan, heparan, heparan sulfate, or a combination thereof.

Next the cells are contacted with the disclosed composition in vitro to increase autophagy of the cells. The cells can be monitored, and the desired cells, for example, cells increase autophagy, can be selected for therapeutic administration.

The cells can be administered to a patient in need thereof. In the most preferred embodiments, the cells are isolated from and administered back to the same patient. In alternative embodiments, the cells are isolated from one patient, and administered to a second patient. The method can also be used to produce frozen stocks of primed cells which can be stored long-term, for later use. In one embodiment hematopoietic or mesenchymal stem cells are isolated from a patient and primed in vitro to provide therapeutic cells for the patient.

3. In Vivo Methods

The disclosed compositions can be used in a method of increasing the autophagy of cells in vivo. In some in vivo approaches, the compositions are administered directly to a subject in a therapeutically effective amount. The amount can be a dosage sufficient to treat, inhibit, or alleviate one or more symptoms of the disorder being treated or to otherwise provide a desired pharmacologic and/or physiologic effect. For example, in some embodiments, the composition for increasing the bioavailability of one or more active isoforms of SDF-1 is administered to a subject in an effective amount to increase the survival of a population of cells in a subject. As discussed in more detail below, the composition can be administered locally or systemically and can be used to increase the survivability or longevity of an endogenous stem cell niche or transplanted cells or grafts. The precise dosage will vary according to a variety of factors such as subject-dependent variables (e.g., age, immune system health, etc.), the disease, and the treatment being effected.

The composition can be incorporated on or into a delivery vehicle such as micro- or nanoparticles, or lipid micelles. The delivery vehicle can increase stability of the composition in vivo, increase targeting to the desired population of cells, or a combination thereof.

B. Diseases to be Treated

1. Method Increasing Graft Survivability

The compositions and methods can be used to increase graft acceptance and survivability in a recipient. Generally the composition is contacted with the cells of the graft to increase its acceptance or survivability after transplant into a recipient. As discussed in more detail below, the cells of the graft or the site of the graft, or a combination thereof can be treated with the composition prior to implantation into the recipient, after implantation into the recipient, or a combination thereof. In a preferred embodiment, cells of the graft are pre-treated with the composition prior to implantation into the recipient.

The compositions and methods can be used to increase the acceptance, longevity, or survivability of various transplants and grafts. In a preferred embodiment, the compositions and methods are used to increase the acceptance, longevity, or survivability of a transplant or graft that includes mesenchymal stem cells, or MSCs. In some embodiments, the transplant is a bone marrow transplant.

The transplanted material can be cells, tissues, organs, limbs, digits or a portion of the body, preferably the human body. The transplants are typically allogenic or xenogenic. The compositions can be administered systemically or locally by any acceptable route of administration. In some embodiments, the compositions are administered to a site of transplantation prior to, at the time of, or following transplantation. In one embodiment, the compositions are administered to a site of transplantation parenterally, such as by subcutaneous injection.

In other embodiments compositions are administered directly to cells, tissue or organ to be transplanted ex vivo. In one embodiment, the transplant material is contacted with composition prior to transplantation, after transplantation, or both.

The transplant material can be treated with enzymes or other materials that remove cell surface proteins, carbohydrates, or lipids that are known or suspected in being involved with immune responses such as transplant rejection.

a. Cells

Populations of any types of cells can be transplanted into a subject. The cells can be homogenous or heterogenous. Heterogeneous means the cell population contains more than one type of cell. Exemplary cells include progenitor cells such as stem cells and pluripotent cells which can be harvested from a donor and transplanted into a subject. The cells are optionally treated prior to transplantation as mention above.

b. Tissues

Any tissue can be used as a transplant. Exemplary tissues include skin, adipose tissue, cardiovascular tissue such as veins, arteries, capillaries, valves; neural tissue, bone marrow, pulmonary tissue, ocular tissue such as corneas and lens, cartilage, bone, and mucosal tissue. The tissue can be modified as discussed above.

c. Organs

Exemplary organs that can be used for transplant include, but are not limited to kidney, liver, heart, spleen, bladder, lung, stomach, eye, tongue, pancreas, intestine, etc. The organ to be transplanted can also be modified prior to transplantation as discussed above.

One embodiment provides a method of inhibiting or reducing chronic transplant rejection in a subject by administering an effective amount of nanolipogel particles to inhibit or reduce chronic transplant rejection relative to a control.

2 Methods of Increasing Recovery from Injury

The compositions and methods disclosed herein can be used to increase the rate of recovery from injury or improve injury repair.

a. Acute Injuries

For example, the compositions and methods disclosed herein can be used to increase recovery from or repair of acute injuries such as trauma, wounds, fractures, defects, and surgery. The compositions and methods can be used for any acute injury where protection and increased survival of exogenous or endogenous stem cells would improve the rate or quality of recovery or repair. For example, the compositions can be used to protect endogenous MSCs and osteoprogenitor cells allowing them to be available to mediate repairs. The compositions can be used to increase the bioavailability of active SDF-1, for example, by reducing degradation of endogenous SDF-1 by serum and tissue DPP4, or other proteases at the site of injury or repair

The compositions can be administered locally or topically to the site of injury, or coated or impregnated into a bandage used at the site of injury.

It is believed that the compositions disclosed herein are particularly effective to enhancing the survivability of osteogenic progenitor cells, which increases the pool of cells available for mediating bone repair. Accordingly, the compositions and methods are used to improve repair or recovery of bone-specific injuries including, bone fractures, bone defects, or bone-related surgeries or transplants by increasing the longevity or survivability of MSCs. Exemplary surgeries include, but not limited to, dental implants, joint replacement (e.g. hip & knee), ligament repair, and spinal fusion surgeries.

b. Chronic Injuries and Diseases

The compositions and methods disclosed herein can be used to increase recovery from or repair of, or reduce progression of injuries or diseases. In some embodiments the compositions and methods are used to treat tissues undergoing stress or ischemia or other physiologic processes that alter SDF-axis molecule availability or functionality. For example, the compositions and methods can be used to improve neurological injury applications of transplanted MultiStem cells for adult stroke and infant/child hypoxia ischemia.

The compositions and methods can also be used to treat inflammatory diseases, or a symptom thereof. Chronic inflammatory diseases of almost any cause are associated with bone loss (reviewed by Hardy and Cooper, J. Endochrinol., 201:309-320 (2009)). Bone loss is due to direct effects of inflammation, poor nutrition, reduced lean body mass, immobility and the effects of treatments, especially glucocorticoids. Inflammatory disease can increase bone resorption, decrease bone formation but most commonly impacts on both of these processes resulting in an uncoupling of bone formation from resorption in favour of excess resorption. Accordingly, the compositions and methods can be used to reduce or inhibit the impact of inflammatory diseases on bone by increasing the survivability of osteogenic progenitor cells such as MSCs.

Exemplary inflammatory diseases that are associated with bone loss include inflammatory joint disease (best exemplified by rheumatoid arthritis), inflammatory bowel disease (e.g. Crohn's disease or ulcerative colitis), coeliac disease, lung inflammation (asthma, chronic obstructive pulmonary disease, alveolitis), renal disease (nephritis, vasculitis) and disease affecting nerve and muscle (myositis, inflammatory neuropathy) (Hardy and Cooper, J. Endochrinol., 201:309-320 (2009)).

In a particular embodiment, the inflammatory disease is osteoarthritis.

In another embodiment, the compositions and methods are used to increase survival of stem cells populations before, during, or after a chemotherapeutic regime.

3. Methods of Reducing the Effects of Aging

The compositions and methods disclosed herein can be used to reduce one or more effects of aging. It is known that autophagy is reduced in several different tissues with age. For example, studies show that autophagy declines in the brain and bone (including human bone marrow MSCs that give rise to bone forming cells). There is also evidence that it declines in other stem cell populations with age. As such, the survival of stem and progenitor populations with age, or following survival challenges, may be reduced with a subsequent loss of stem cells, including “adult” stem cells in stem cell niches and tissue progenitor/stem cells resulting in age-associated declines in tissue maintenance and repair. Indeed, loss of stem cells may in-part underlie numerous elements related to a decline in tissue repair capacity throughout the body in aging. Accordingly, in some embodiments the compositions are administered in a prophylactically or therapeutic effective amount to increase survival of stem cells, particularly endogenous stem cells resident in stem cell niches. The administration can be effective to reduce the progression of one or more symptoms or effects of aging over time.

MSC population declines with age (in both males and females) and this decline is proposed to be important in the development and progression of age-associated osteoporosis. Furthermore, links between estrogen receptor signaling and SDF-1 expression indicate that SDF-1 may be important in preventing osteoporosis following menopause or estrogen loss. Therefore, the composition can be administered to a subject in an effective amount to treat or prevent osteoporosis, or one or more symptoms thereof.

### EXAMPLES

### Example 1

### Increased Expression of SDF-1β does not Increase Stem Cell Profilteration

### Materials and Methods

Isolation and Culture of BMSCs

BMSCs were derived from 18-month-old male C57BL/6J mice at the Georgia Health Sciences University Stem Cell Core Facility. Male C57BL/6 mice were purchased from the National Institute on Aging (Bethesda, Md., USA) aged rodent colony. Animals were maintained at the Georgia Health Sciences University—Division of Laboratory Animal Services Facility. All aspects of the animal research were conducted in accordance with the guidelines set by the Georgia Health Sciences University Institutional Animal Care and Use Committee (GHSU-IACUC) under a GHSU-IACUC approved Animal Use Protocol.

The BMSC isolation process, retroviral transduction to express Green Fluorescent Protein (GFP), and clonal selection have been described previously (Herberg, et al., Tissue Eng. Part A, 19:1-13 (2013)); Zhang, et al., Journal of Bone and Mineral Research, 23:1118-1128 (2008)); Zhang, et al., Journal of Biological Chemistry, 283:4723-4729)). In brief, six mice were euthanized by CO₂ overdose followed by thoracotomy. Whole bone marrow aspirates were flushed from femora and tibiae and BMSCs isolated by negative immunodepletion using magnetic microbeads conjugated to anti-mouse CD11b (cat#558013), CD45R/B220 (cat#551513) (BD Biosciences Pharmingen, San Diego, Calif., USA), CD11c, and plasmacytoid dendritic cell antigen (PDCA)-1 (cat#130-092-465) (Miltenyi Biotec, Bergisch Gladbach, Germany) followed by positive immunoselection using anti-stem cell antigen (Sca)-1 microbeads (cat#130-092-529) (Miltenyi Biotec) according to the manufacturer's recommendations. Enriched BMSCs were labeled with GFP, and maintained in Dulbecco's Modified Eagle Medium (cat#10-013) (DMEM; Cellgro, Mediatech, Manassas, Va., USA) supplemented with 10% heat-inactivated fetal bovine serum (cat#S11150) (FBS; Atlanta Biologicals, Lawrenceville, Ga., USA). As described in detail, clone 2 was used as the parental cells for further genetic modification with the Tet-Off system at 70-80% confluence.

Genetic Modification of BMSCs for Conditional Expression of SDF-1β

BMSCs were transduced with retroviral Tet-Off expression vectors. The sequential protocol of retrovirus production, two-step infection, and selection to generate double-stable Tet-Off-SDF-1β BMSCs and Tet-Off-EV control BMSCs has been described previously (Herberg, et al., Tissue Eng Part A, 19:1-13 (2013)). In brief, 293GPG packaging cells (Ory, et al., Proc. Natl. Acad. Sci. USA, 93:11400-11406 (1996)) were transfected at passage 8 with retroviral Tet-Off expression vectors containing the SDF-1β coding sequence, or empty control (cat#632105) (Clontech Laboratories, Mountain View, Calif., USA). BMSCs (clone 2) were infected at passage 10 with 2 ml of the respective retroviral supernatant containing 4 μg/ml polybrene (cat#H9268) (Sigma-Aldrich, St. Louis, Mo., USA) and 100 ng/ml doxycycline (cat#D9891) (Dox; Sigma-Aldrich) followed by selection with 400 μg/ml G418 (cat#091672548) (MP Biomedicals, Solon, Ohio, USA) and 2.5 μg/ml puromycin (cat#P8833) (Sigma-Aldrich). Clonally selected parental BMSCs and Tet-Off-modified BMSCs were shown to retain their multipotent differentiation potential, including osteogenic potential, over more than 10 passages both in vitro and in vivo upon transplantation (see Herberg, et al., Tissue Eng Part A, 19:1-13 (2013) and unpublished data). Genetically engineered BMSCs were maintained in DMEM supplemented with 10% Tet-FBS (cat#631106) (Clontech), 400 μg/ml G418, and 2.5 μg/ml puromycin. For in vitro experiments, cells at passage 16 were plated at 2.5×10³ cells/cm² and then treated with Dox starting the next day. The medium was exchanged daily. To induce cell death, genetically engineered BMSCs were incubated with 1.0 mM H₂O₂ or vehicle control for 6 h.

Cell Proliferation

Cell proliferation of BMSCs in normal growth medium was measured over the course of 7 d using the Vybrant® MTT Cell Proliferation Assay Kit (cat#V13154) (Molecular Probes, Eugene, Oreg., USA) according to the manufacturer's recommendation. The assay involves the conversion of the water-soluble MTT (3-(4,5-dimethylthiazol-2-yl)-2,5-diphenyltetrazolium bromide) to an insoluble formazan, which is then solubilized using DMSO and its absorbance measured at 540 nm.

### Results

In the Tet-Off system, Dox prevents binding of the Tet-controlled transactivator to the Tet-promoter on the response vector and thus suppresses transcription of the downstream gene of interest, in our case SDF-1β (Gossen, et al., Annual Review of Genetics, 36:153-173 (2002)). It was previously shown that SDF-1β mRNA expression by Tet-Off-SDF-1β BMSCs was 30-fold increased compared to controls and this increase was accompanied by a similar augmentation in SDF-1β protein levels (Herberg, et al., Tissue Eng Part A, 19:1-13 (2013). Controls included an internal control (+Dox) and a second external empty vector control, which have been shown to possess comparable levels of SDF-1 splice variant expression and downstream effects when subjected to osteogenic differentiation (Herberg, et al., Tissue Eng Part A, 19:1-13 (2013).

A potential role of the SDF-1/CXCR4 signaling axis in maintaining proliferation and survival of stem cell populations in the BM was discussed in (Kucia, et al., J. Mol. Histol., 35:233-245 (2004)) and SDF-1(α) pre-treatment or over-expression was described to promote the proliferation and survival of rat and human BMSCs after re-oxygenation following H₂O₂-treatment or exposure to the apoptosis-inducing cytokine IL-4 (Liu, et al., Protein Cell, 2:845-854 (2011); Kortesidis, et al., Blood, 105:3793-3801 (2005)).

An experiment was designed to determine if SDF-1β enhances BMSCs proliferation and survival, and specifically if survival was through effects of SDF-1β on apoptotic and/or autophagic mechanisms. BMSCs were cultured in normal growth medium and the absorbance of DMSO-solubilized MTT formazan was measured at 540 nm after 1, 3, and 7 days (FIG. 1). No differences in cell proliferation were found in Tet-Off-SDF-1β BMSCs compared to Dox-suppressed and Tet-Off-EV controls over the course of 7 d.

In summary, it was determined that increasing SDF-1β above basal levels had no effect on BMSC proliferation over the course of 7 d compared to controls.

### Example 2

### Increased Expression of SDF-1β Improves Stem Cell Viability

### Materials and Methods

Cell and Nuclear Morphology

Morphological changes of BMSCs in response to H₂O₂ treatment were visualized by phase contrast microscopy. Furthermore, the chromatin dye Hoechst 33342 was used to assess alterations in the nuclear morphology. Cells were washed with PBS, fixed with methanol for 10 min at −20° C., and stained with 5 μg/ml Hoechst 33342 (cat#62249) (Pierce, Thermo Fisher Scientific) for 30 min at room temperature. BMSCs undergoing cell death were visualized by standard phase contrast and fluorescence microscopy using an inverted microscope (Carl Zeiss, Jena, Germany) equipped with an Exfo X-Cite 120 fluorescence lamp (Lumen Dynamics, Mississauga, Ontario, Canada).

Cell Viability

The viability of BMSCs in response to H₂O₂ treatment was analyzed using standard trypan blue exclusion staining Cells were washed with PBS, lifted with trypsin/EDTA, and resuspended with normal growth medium. Following 1:5 dilution, BMSC suspensions were mixed with an equal volume of 0.4% trypan blue staining solution (cat#15250061) (Gibco, Invitrogen), and counted in 5 inner squares using a hemacytometer with cover slip (Hausser Scientific, Horsham, Pa., USA).

### Results

The role of SDF-1β in protecting BMSCs from cell death was investigated. The concentration of H₂O₂ necessary to deplete approximately 50-60% of BMSCs was established previously in a series of dose-response and time course studies. BMSCs were incubated with 1.0 mM H₂O₂ for 6 h before the cell and nuclear morphology were assessed by standard phase contrast microscopy and Hoechst 33342 staining. No differences in cell and nuclear morphology were found among all vehicle-treated control groups. In contrast, SDF-1β markedly protected Tet-Off-SDF-1β BMSCs from H₂O₂-induced cell death relative to Dox-suppressed and Tet-Off-EV controls. Overall more live cells retaining the typical spindle-shaped BMSC morphology with normal round nuclei were observed compared to controls showing substantial cell loss/shrinkage and condensed nuclei, indicative of apoptosis.

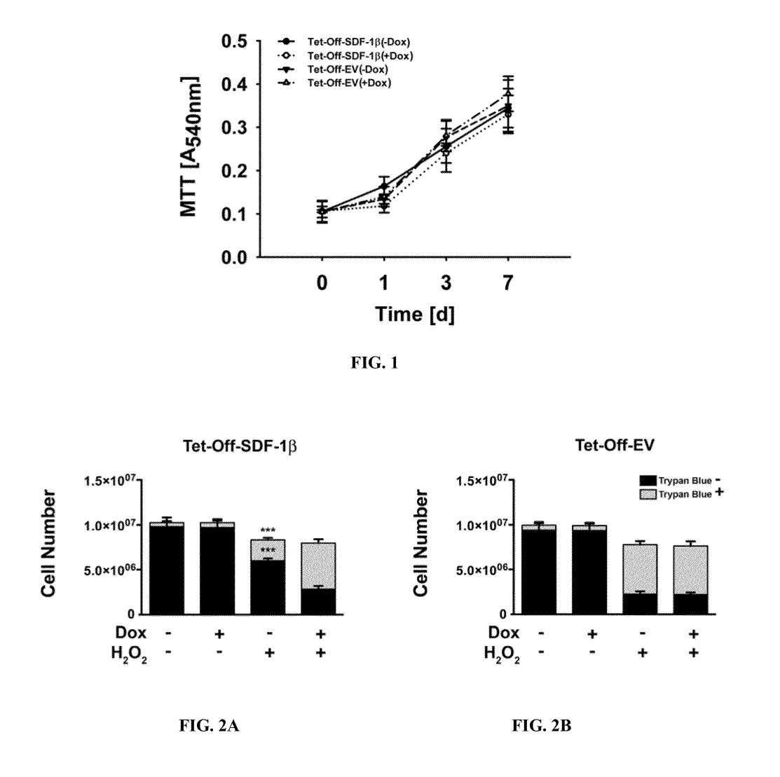

The total number of surviving cells were quantified using a standard trypan blue staining protocol (FIG. 2). In agreement with previous results, no differences in the number of trypan blue negative and positive BMSCs were found among vehicle control groups. In contrast, SDF-1β in Tet-Off-SDF-1β BMSCs significantly increased the number of surviving cells and decreased the number of dying cells in response to H₂O₂ treatment compared to Dox-suppressed (FIG. 2A) and Tet-Off-EV controls (FIG. 2B) (trypan blue negative: Tet-Off-SDF-1β: −Dox, 6.0×10⁶±2.7×10⁵ cells, +Dox, 2.8×10⁶±3.6×10⁵ cells; Tet-Off-EV: −Dox, 2.3×10⁶±3.2×10⁵ cells, +Dox, 2.2×10⁶±2.5×10⁵ cells; p<0.0001; trypan blue positive: Tet-Off-SDF-1β: −Dox, 2.3×10⁶±2.5×10⁵ cells, +Dox, 5.2×10⁶±4.4×10⁵ cells; Tet-Off-EV: −Dox, 5.5×10⁶±4.1×10⁵ cells, +Dox, 5.4×10⁶±5.4×10⁵ cells; p<0.0001).

In summary, it was determined that increasing SDF-1β above basal levels had no effect on BMSC proliferation (Example 1), increasing SDF-1β significantly protected BMSCs from H₂O₂-induced cell death resulting in increased numbers of surviving cells relative to controls, which also retained their typical spindle-shaped morphology and normal round nuclei. Together, these results indicate that SDF-1β-mediated protection of BMSCs against cell death could be independent from potential effects on cell proliferation.

### Example 3

### Increased Expression of SDF-1β Reduces Cell Death and Increases Autophagy of Stem Cells

### Materials and Methods

Western Blotting

Whole cell lysates of BMSCs in response to H₂O₂ treatment were prepared in Complete Lysis-M EDTA-free buffer containing protease inhibitors (cat#04719964001) (Roche Diagnostics, Indianapolis, Ind., USA). Equal amounts (20 μg) of protein lysates were subjected to SDS-PAGE using 10% NuPAGE® Bis-Tris gels (cat#NP0315BOX) (Invitrogen) and transferred to 0.2 μm PVDF membranes (cat#ISEQ00010) (Millipore, Billerica, Mass., USA). Membranes were blocked with 5% non-fat milk in TBST. Apoptosis and autophagy markers were detected using specific primary antibodies (anti-poly(ADP-ribose) polymerase (PARP) (cat#9532), anti-cleaved PARP (cat#9544), anti-cleaved caspase-3 (cat#9664): Cell Signaling Technology, Danvers, Mass., USA; anti-beclin 1 (cat#ab16998): Abcam, Cambridge, Mass., USA; anti-LC3B-II (cat#R-155-100): Novus Biologicals, Littleton, Colo., USA; anti-β-actin (cat#A1978): Sigma-Aldrich, St. Louis, Mo., USA) followed by HRP-conjugated secondary antibodies (D-anti-Rb cat#711-035-152; D-anti-Ms cat#715-035-150) (Jackson ImmunoResearch, West Grove, Pa., USA). Bound antibodies were visualized with the ECL detection system (cat#34080) (Pierce, Thermo Fisher Scientific) on autoradiography film (cat#E3018) (Denville Scientific, Metuchen, N.J., USA). The intensity of immunoreactive bands was quantified using Photoshop CS4 v11.0 (Adobe Systems, San Jose, Calif., USA).

Statistical Analysis

Experiments were performed three independent times (n=3-6). All data are expressed as means±SD. Analysis of variance (ANOVA) followed by Tukey's or Bonferroni's post hoc test were used to determine mean differences between groups. Null hypotheses were rejected at the 0.05 level. Data were analyzed using GraphPad Prism 5.0 software (GraphPad Software Inc., La Jolla, Calif., USA).

### Results

Upon binding to its cognate receptor CXCR4, SDF-1 has been implicated in modulating the survival-enhancing PI3-kinase/Akt and MAP-kinase/Erk1/2 signaling pathways (Mangi, et al., Nat. Med., 9:1195-1201 (2003); Xu, et al., J. Cell Biochem., 103:256-269 (2008); Choi, et al., Stem Cells Dev., 17:725-736 (2008); Zhang, et al., J. Mol. Cell Cardiol., 48:1060-1070 (2010)), which can be blocked by the specific CXCR4 antagonist AMD3100 (Liu, et al., Protein Cell, 2:845-954 (2011). In addition, it was shown that SDF-1 can increase the levels of anti-apoptotic Bcl-2 and decrease the levels of pro-apoptotic Bax (Liu, et al., Protein Cell, 2:945-854 (2011); Teicher, et al., Clin. Cancer Res., 16:2927-2931 (2010)).

Caspase activation plays a central role in the execution and completion of apoptosis. In particular, caspase-3 is critical during early apoptosis as it is involved in the proteolytic cleavage/activation of many key proteins such as PARP and other caspases (Fernandes-Alnemri, et al., J. Biol. Chem., 269:30761-30764 (1994); Porter, et al., Cell Death Differ, 6:99-104 (1999)).

Stressors can induce either apoptosis or autophagy in a context-dependent fashion. In some cases, a mixed phenotype of apoptosis and autophagy can be detected (Maiuri, et al., Nat. Rev. Mol. Cell Biol., 8:741-752 (2007)). The induction of autophagy utilizes two ubiquitin-like conjugation systems as part of the vesicle elongation process. One pathway involves the covalent conjugation of Atg12 to Atg5 and the second pathway comprises the conjugation of phosphatidylethanolamine to LC3/Atg8. Lipid conjugation then leads to the conversion of the soluble form of LC3-I to the autophagosome-associated form LC3-II (Tanida, et al., J Biochem Cell Biol, 36:2503-2518 (2004)). Another key player involved in the onset of autophagy is beclin 1. Like other BH3-only proteins, beclin 1 interacts with anti-apoptotic multi-domain proteins of the Bcl-2 family via its BH3 domain, and this interaction can be competitively disrupted to liberate beclin 1 and stimulate autophagy (Maiuri, et al., EMBO J., 26:2527-2539 (2007); Kessel, et al., Cancer Lett., 249:294-299 (2007); Daido, et al., Cancer Res., 64:4286-4293 (2004); Hamacher-Brady, et al., Cell Death Differ, 14:146-157 (2007); Oberstein, et al., J. Biol. Chem., 282:13123-13132 (2007); Maiuri, et al., Autophagy, 3:374-376 (2007); Nobukuni, et al., Curr. Opin. Cell Biol., 19:135-141 (2007)).

Despite the well-characterized signaling pathways involved, very little is known about the role of autophagy in MSC maintenance and differentiation. In a recent study it was shown that MSCs possess high levels of basal autophagy and that suppression of autophagy through knockdown of Bcl-2-xL dramatically impairs the survival and differentiation capacities of human MSCs (Oliver, et al., Stem Cells Dev., 21:2779-2788 (2012)). Furthermore, the activation of autophagy has been linked to protection of MSCs from hypoxia and serum deprivation through regulating the phosphorylation of mTOR (Lee, et al., J. Cell Biochem., (2007); Zhang, et al., Stem Cells Dev., 21:1321-1332 (2012)). In addition, a genome-wide siRNA screening revealed that under normal homeostatic conditions upregulation of autophagy requires the type III PI3-kinase, but not inhibition of mTORC1 (Lipinski, et al., Dev. Cell, 18:1041-1052 (2010)). Positive regulators of cell survival and proliferation including SDF-1/CXCR4 were identified to be involved in regulating autophagy in different cell types (Lipinski, et al., Dev. Cell, 18:1041-1052 (2010)). It is believed that prior to the experiments disclosed herein, a direct link between the survival-enhancing effects of the SDF-1/CXCR4 axis and autophagy in BMSCs had not been established.

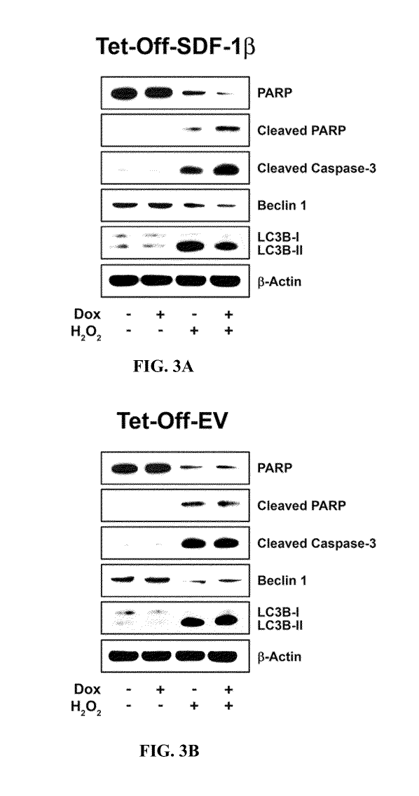

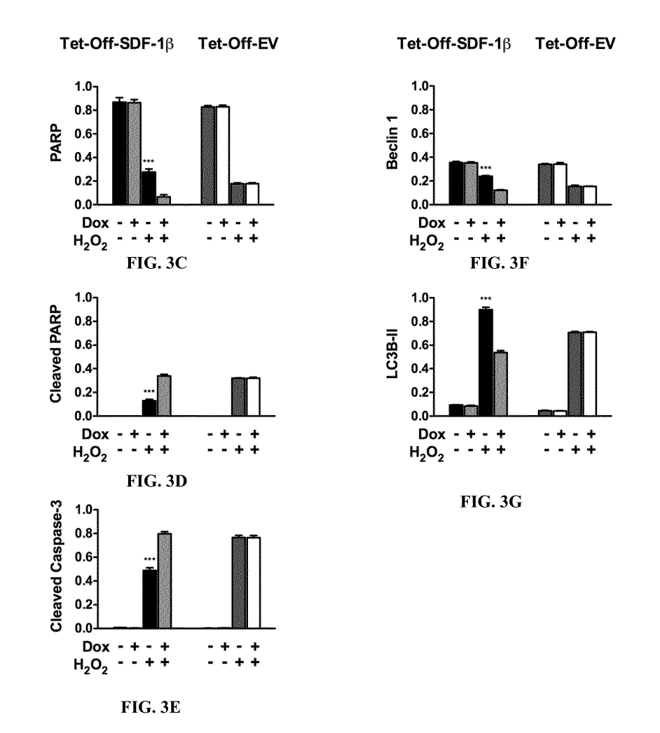

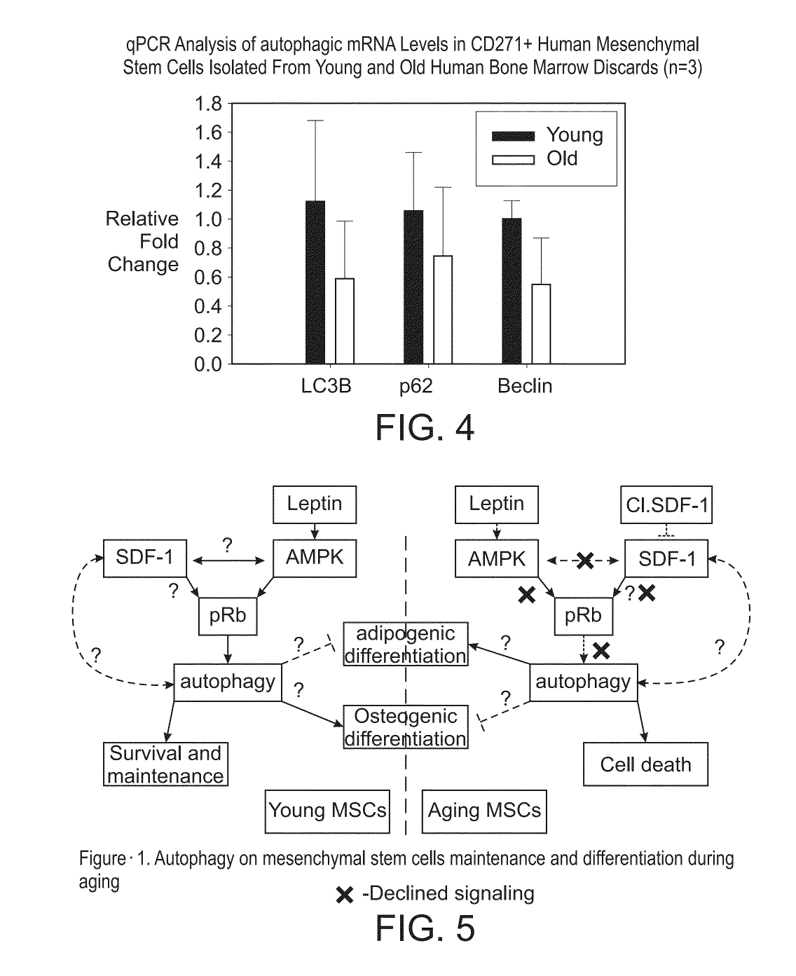

Experiments were designed to determine the cellular mechanisms underlying the SDF-1β-dependent protection from cell death in novel Tet-Off-SDF-1β BMSCs, by focusing on key players involved in two cell death/survival processes, apoptosis and autophagy, in response to oxidative stress. To determine the mechanism of SDF-1β-mediated protection from cell death, key players involved in apoptosis and autophagy were investigated (FIG. 3). Western blot analysis showed that the relative levels of apoptosis markers PARP (FIG. 3C), cleaved PARP (FIG. 3D), and cleaved caspase-3 (FIG. 3E) as well as autophagy markers beclin 1 (FIG. 3F) and LC3B-II (FIG. 3G) were comparable among all vehicle control groups. In contrast, SDF-1β significantly increased the normalized levels of intact PARP, decreased the levels of cleaved PARP and cleaved caspase-3, and increased the levels of beclin 1 and LC3B-II in Tet-Off-SDF-1β BMSCs in response to H₂O₂ treatment (FIGS. 3A-G) relative to Dox-suppressed controls (PARP: −Dox, 0.29±0.01, +Dox, 0.10±0.03; p<0.01; cleaved PARP: −Dox, 0.12±0.01, +Dox, 0.35±0.01; p<0.001; cleaved caspase-3: −Dox, 0.49±0.01, +Dox, 0.80±0.02; p<0.001; beclin 1: −Dox, 0.24±0.01, +Dox, 0.12±0.01; p<0.01; LC3B-II: −Dox, 0.89±0.02, +Dox, 0.53±0.02; p<0.001). No differences were found between H₂O₂-treated Tet-Off-EV control groups (FIGS. 5B-G) (PARP: −Dox, 0.17±0.01, +Dox, 0.16±0.01; cleaved PARP: −Dox, 0.32±0.01, +Dox, 0.30±0.01; cleaved caspase-3: −Dox, 0.75±0.01, +Dox, 0.73±0.01; beclin 1: −Dox, 0.13±0.01, +Dox, 0.15±0.01; LC3B-II: −Dox, 0.70±0.01, +Dox, 0.71±0.01).

In summary, Western blot analysis indicates that SDF-1β over-expression significantly decreased the levels of cleaved caspase-3, resulting in decreased levels of cleaved PARP and, in turn, increased levels of intact PARP relative to controls suggesting that SDF-1β partially blocked caspase-3-dependent apoptosis in BMSCs. The observed SDF-1β-mediated reduction in caspase-3-dependent apoptosis of BMSCs coincided with an increase in autophagy. Western blot analysis revealed that SDF-1β significantly increased the levels of LC3B-II and beclin 1 compared to controls, demonstrating an increase in autophagic markers, and indicating that SDF-1β exerts part of its cell-protection through increasing autophagy in BMSCs. This is believed to be the first report of a direct interaction of the SDF-1/CXCR4 signaling axis, and specifically the SDF-1β isoform, with autophagy in BMSCs.

### Example 4

### Autophage is Reduced in Aged CD271+ Human Mesenchymal Stem Cells

FIG. 4 shows the results of qPCR analysis of autophagic mRNA levels in CD271+ human mesenchymal stem cells isolated from “young” and “old” human bone marrow discards. The results show that expression of markers for autophagy including LC3B, p62, and Beclin are reduced in “aged” subjects.

### Example 5

### SDF-1 Regulates Autophagy in Mesenchymal Stem Cells

### Materials and Methods

Mesenchymal Stem Cells

Low passage MSCs from 18 and 24 month old adult male C57BL/6 mice, procured from the GHSU Institute of Regenerative and Reparative Medicine Stem Cell Core (IRRM-SSC) were used. This age range was chosen based on associated changes in bone formation and mass in the comparatively aged mice (Zhang, et al., Journal of Bone and Mineral Research, 23:1118-1128 (2008)).

Active Agents

Recombinant active SDF-1β (i.e. full length amino acids 1-72; 100 ng/mL; Peprotech), Recombinant Mouse DPPIV/CD26 enzyme (0.01 μg; R&D) cleaved (Cl.) SDF-1β (aa3-72; 100 ng/mL) (Christopherson, et al., Blood, 101:4680-4686 (2003)), and AMD3100 (400 μM; R&D), an antagonist of SDF-1/CXCR4 axis (Liu, et al., Protein & Cell, 2:845-854 (2011)).

### Results

FIG. 5 is a diagram illustrating the proposed role SDF-1 signaling pathway in regulating autophagy at the transcriptional and protein level in MSCs.

Redistribution of LC3B from cytosol (18 kDa) to an autophagosome (16 kDa) is an indication of autophagy induction. P62, also known as sequestosome 1/SQSTM1, is a ubiquitously expressed protein, best characterized for selective autophagy, directly interacts with LC3B on the phagophore through the LC3-interacting region and then degraded. Impairment of autophagy is accompanied by accumulation of p62 and LC3B for a prolonged period (Periyasamy-Thandavan, et al., Autophagy, 5:19-35 (2010); Periyasamy-Thandavan, et al., Renal Physiology, 297:F244-256 (2009); S. Periyasamy-Thandavan, et al., Kidney International, 74:631-640 (2008)). MAPK activation has been documented as a positive regulator during autophagy induction and inhibition of ERK1/2 abrogates induction of autophagy (Dagda, et al., Autophagy, 4:770-782 (2008)).

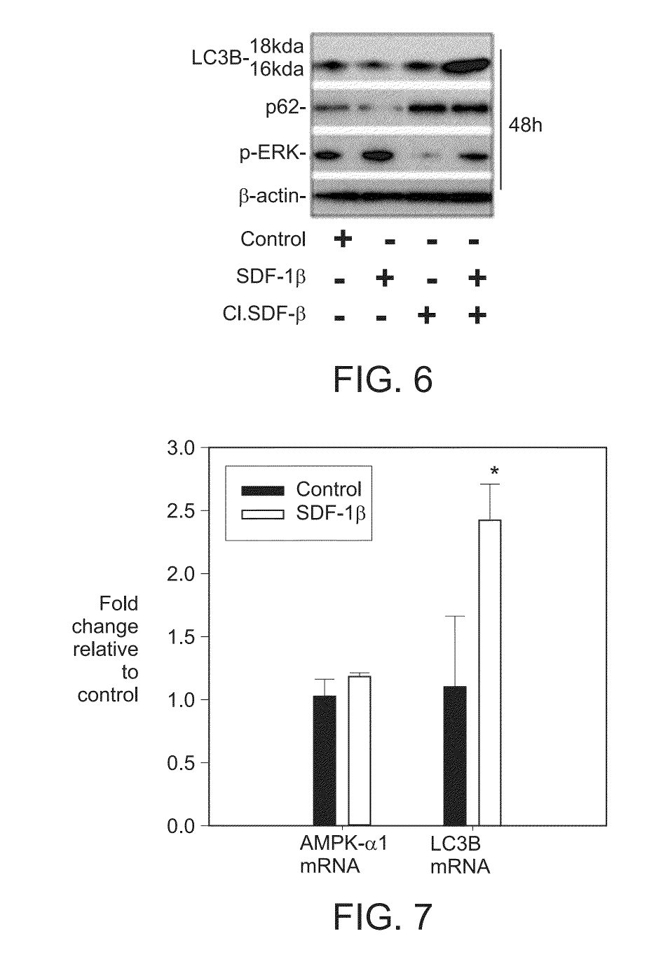

The effect of SDF-1 treatment on autophagy and MAPK pathway either by the use of SDF1β or C1.SDF1β, or their combination in 18 month MSCs was examined. Six hours of SDF-1β treatment led to MAPK pathway activation and induced autophagy by distributing LC3B and further incubation to 24 or 48 h led to gradual disappearance of LC3B and p62 (FIG. 6), an observation that was consistent with the degradation of LC3B and p62 in matured autophagosomes (Periyasamy-Thandavan, et al., Kidney International, 74:631-640 (2008)). Also consistent with previous reports (Sadir, et al., The Journal of Biological Chemistry, 279:43854-43860 (2004); Christopherson, et al., Blood, 101:4680-4686 (2003)), C1.SDF-1β treatment was antagonistic to SDF-1β, leading to attenuated phosphorylation of ERK2 and a buildup in the levels of both LC3B and p62, a sign of defective autophagy.

With RNA isolation, qRTPCR using primers specific to mouse AMPK-α1 and LC3B indicate that SDF-1β regulated autophagy at transcriptional level (FIG. 7).

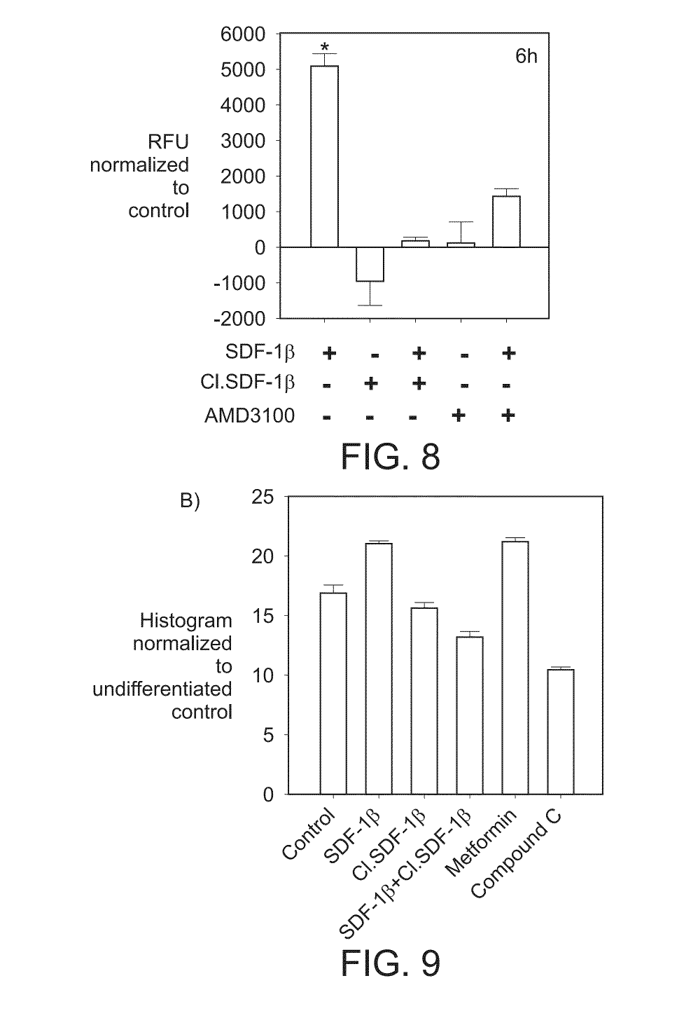

The migration efficiency of 18 month MSCs in response to SDF-1β, its cleaved form and AMD3100, as a positive control, was tested using a transwell migration system. FIG. 8 shows that MSCs exerted strong migrating potential in the presence of SDF-1β relative to control but the migration was suppressed by both c1.SDF-1β and AMD3100. These data show that blockade of autophagy attenuate the migration capacity of MSCs.

To determine the influence of modulating SDF-1 signaling in osteogenic differentiation of 18 month MSCs, the calcium deposition at the terminal stages of differentiation was measured. When compared to control cells maintained in culture medium only, SDF-1β treatment showed increased Alizarin red staining for calcium by ˜20 fold (FIG. 9A). However, the SDF-1β stimulated calcium-staining was reduced by ˜8 fold (FIG. 9B) with co-treatment with C1.SDF-10. Collectively, these data indicate that perturbing SDF-1 signaling in MSCs affected their osteogenic differentiation via defective autophagy.

### Example 6

### Metformin Treatment Increases Autophagy in Mesenchymal Stem Cells

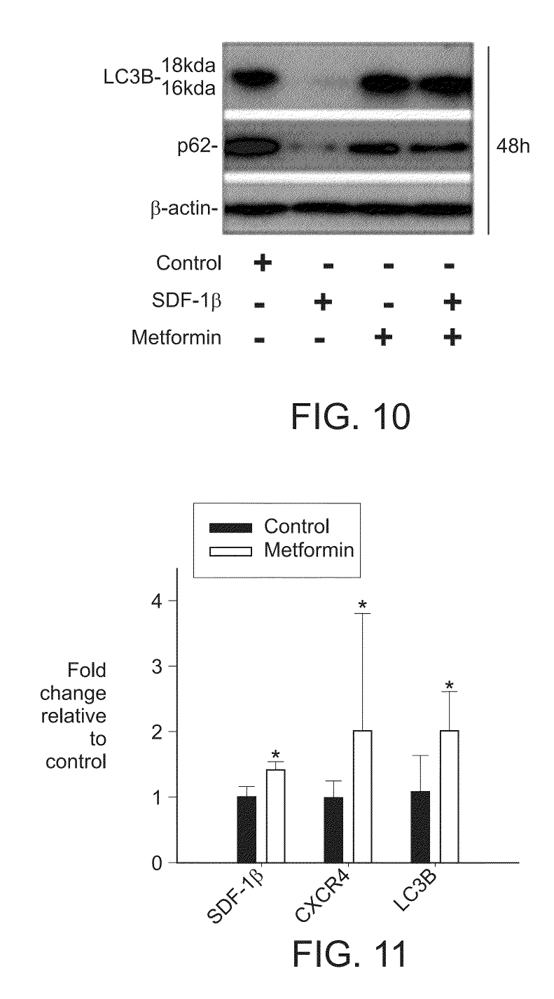

The effect of metformin treatment on autophagy and osteogenic gene expression, with or without SDF1β, was examined in 18 month and 24 month old MSCs. Six hours of metformin treatment induced autophagy by changing the distribution of LC3B. Further incubation to 24 or 48 h led to the gradual reduction of LC3B and p62 (FIG. 10). qRTPCR measurement of SDF1β, CXCR4 and LC3B indicated that AMPK regulates osteogenic and autophagy genes at the transcriptional level (FIG. 11).

In osteogenic differentiation assays metformin treatment showed increased Alizarin red staining, a marker for osteogenic mineralization (calcium deposition) by ˜20 folds (FIG. 9A). In contrast, Compound C, a pAMPK inhibitor, significantly reduced osteogenic differentiation of MSCs (Alizarin red staining) (FIG. 9B).

Collectively, these data indicate that the pAMPK signaling pathway in MSCs effects SDF-1, autophagy and osteogenic differentiation.

### Example 7

### SDF-1 in Bone Marrow Fluid is Less Biologically Active with Age

Materials and Methods

BM supernatant from 3 or 18 month old mice were normalized by dilution with saline to yield 150 pg/ml of SDF-1 as determined by ELISA. The normalized BM supernatant was added to the lower chamber and the cells (either pretreated with AMD3100 or not) were permitted to migrate for 6 hours.

Results

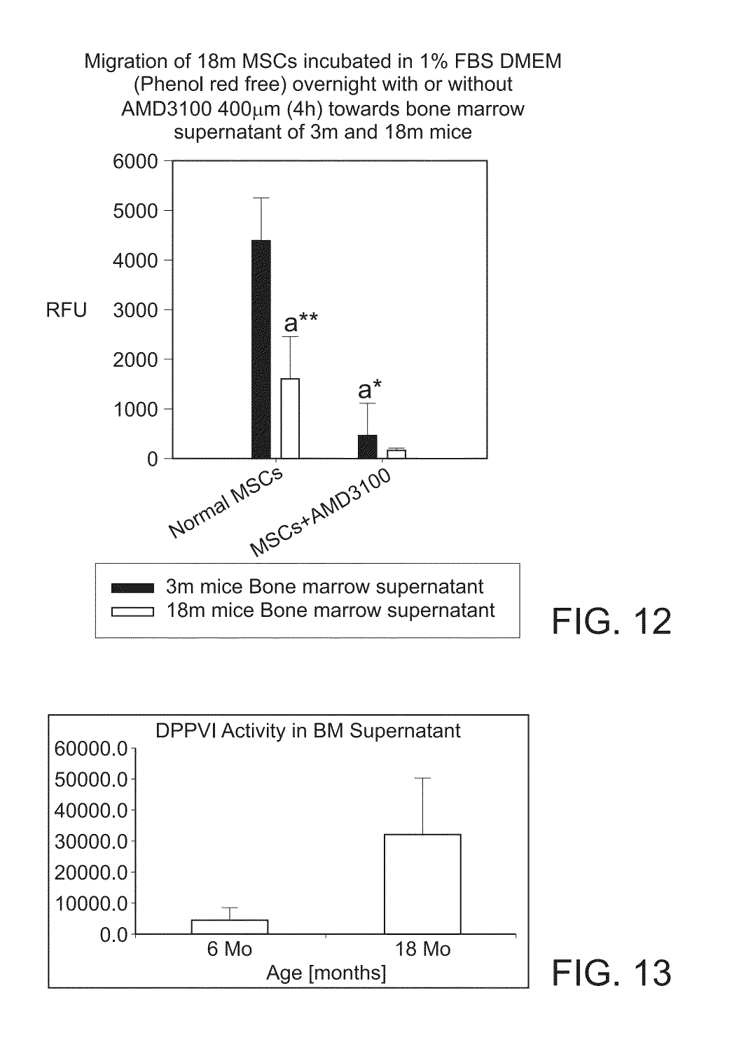

The results presented in FIG. 12 demonstrate that SDF-1 from the BM interstitial fluid of aged mice has a reduced ability to induce BMSC migration. The supernatant from the older mice, despite having equal amounts of SDF-1 compared to the younger mice had significantly less migration, further AMD3100 pretreatment blocked all migration for both age groups, demonstrating that the cell migration was dependent on CXCR4 signaling. This observation supports the idea that the BM supernatant from the older mice contains SDF-1 with reduced bioactivity relative to the young mice, and is in accord with the idea that the regulatable form of DPP4 (Christopher, et al., Blood, (2009); Jin, et al., Bone Marrow Transplant, 42(9):581-588 (2008); Petit, et al., Nature Immunology, 3(7):687-694 (2002); Semerad, et al., Blood, 106(9):3020-3027 (2005)) is more active with age in the BM leading to increased DPP4-cleaved SDF-1 (i.e. CXCR4 binding, but inactive SDF-1), which is responsible for reduced CXCR4 signaling in the aged BM microenvironment.

As shown in FIG. 13 direct measurement of DPP4 activity demonstrates that it is increased in the BM interstitial fluid in aged mice by over seven fold relative to young mice.

### Example 8

### miRNAs Regulate SDF-1 Expression

Based on the microarray data analysis and it was discovered that miRs 29a, 141, 144, 200a, 200c, and 1244 were upregulated in aged BMSC compared to young BMSC. Database analysis revealed SDF-1 as a predicted gene target of these miRNAs.

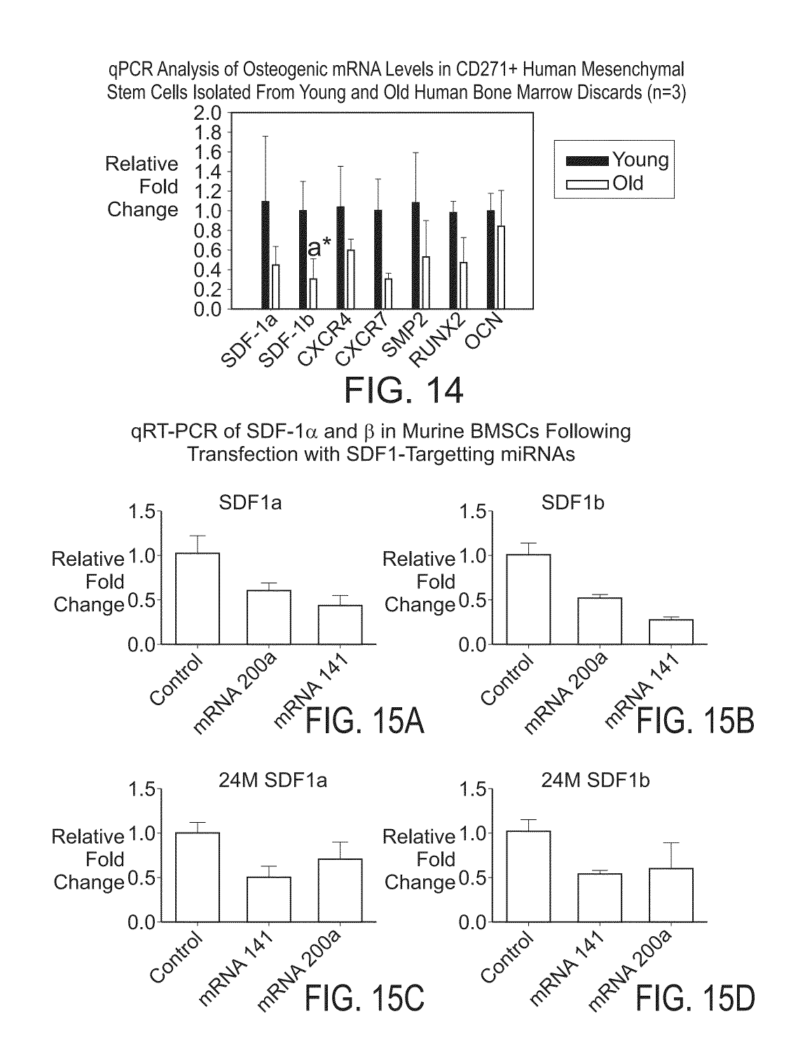

qRT-PCR was performed on mRNA isolated from the same human BMSCs used in the microarray study, with “young” subjects (29-41 years of age, n=3) and “old” subjects (64-73 years of age n=4). mRNA for the SDF-1 axis and osteogenic genes was quantified as described in Herberg et al, (Herberg, et al., Tissue Engineering Part A., (2012)). As shown in FIG. 14 both isoforms of SDF-1 and both receptors show an apparent reduction in mRNA with age, this was also seen for BMP2 and RUNX2. Differences in the expression of SDF-1β was statistically significant.

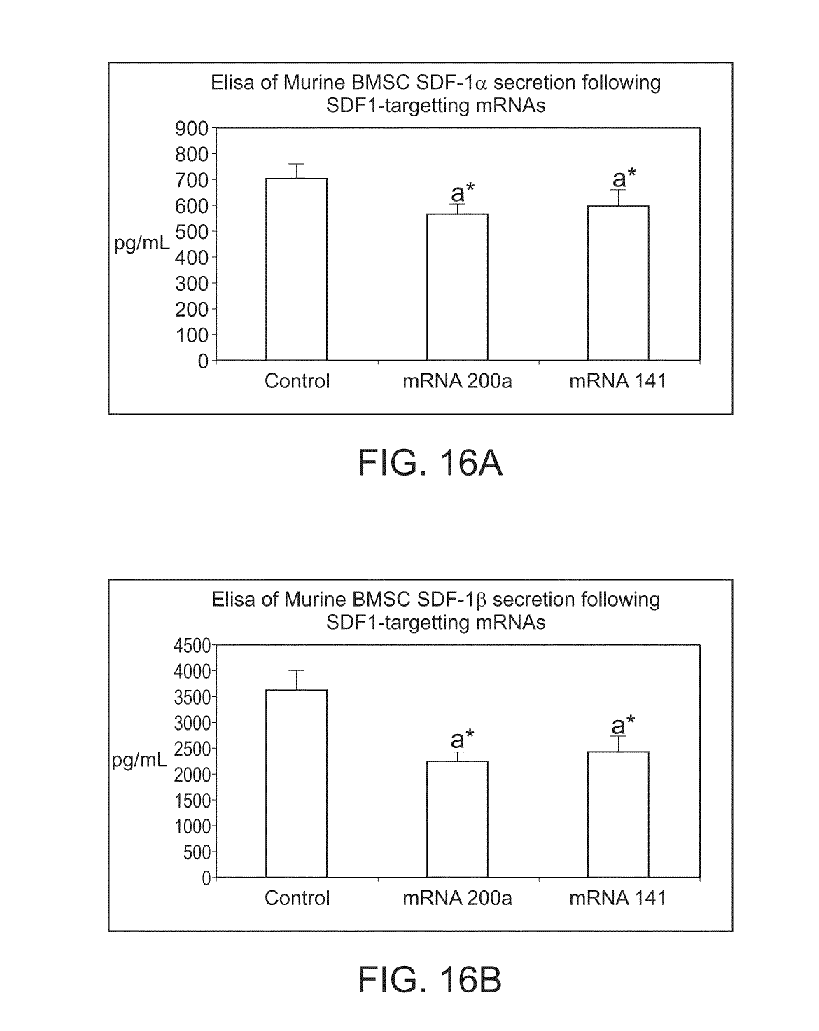

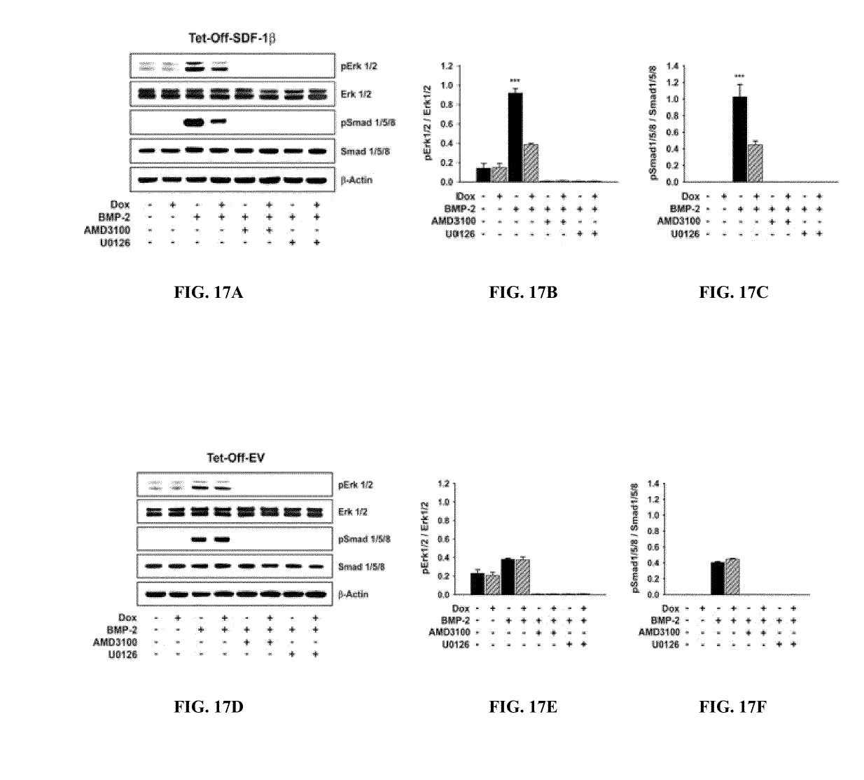

The miRs 141 and 200a were reported to be down-regulated by BMP2 and miRs 144 and 200c were reported to be up-regulated following oxidative stress (Itoh, et al., J. Biol. Chem., 284(29):19272-19279 (2009); Magenta, et al., Cell Death Differ, 18(10):1628-1639 (2011)). These mRNAs were assessed for their potential to reduce SDF-1 mRNA and protein expression by transfecting mimics (mirVana mimics with Lipofectamine, Life Technologies) into murine BMSCs (passage 3 provided by Core C) isolated from 6 or 24 month old mice. The TargetScan database was used to confirm that these miRNAs are shared by humans and mice and that the human and murine SDF-1 3′UTRs also shared miRNA binding sites coding for these miRNAs. In all cases the miRNA mimics decreased SDF-1α & β mRNA levels in the cells (FIGS. 15 and 16). Additionally, two of these miRNAs (141 & 200a) were then selected to transfect murine BMSCs to assess their effect on SDF-1α & β protein expression by ELISA. In both cases SDF-1 levels were decreased. This in vitro data supports the idea that SDF-1 levels in vivo are altered by SDF-1 targeting miRNAs and is consistent with in vivo identification of SDF-1-targeting miRNAs increasing in “old” human BMSCs, as well as the reduced SDF-1 mRNA expression levels seen in “old” human BMSCs.

### Example 9

### SDF-1β Mediates BMSC Function, and Survival

### Materials and Methods

BMP receptor signaling was assessed by Western blot. Serum-starved BMSCs were pretreated with 600 μM AMD3100, 50 μM U0126, or vehicle for 4 h prior to stimulation with 300 ng/ml BMP-2 for 30 min. Whole cell lysates were subjected to SDS-PAGE, electroblotted onto PVDF membranes, and probed for (p)Erk1/2 and (p)Smad1/5/8. Transwell migration assays were performed using conditioned media from genetically engineered BMSCs in lower chambers. Serum-starved Jurkat cells or BMSCs at 5.0-8.0×10⁵ cells/ml in upper chambers were allowed to migrate across the 8-μm membranes for 4-8 h prior to quantifying total DNA with CyQuant GR dye at 485/535 nm. Apoptosis was induced with 1.0 mM H₂O₂ for 6 h. Cell death was quantified by trypan blue staining and Western blot analysis of cleaved caspase-3 and PARP. Autophagy was evaluated by Western blot for LC3-II.

### Results

Recently, genetically engineered bone marrow-derived mesenchymal stem cells (BMSCs) that conditionally overexpress SDF-1β, the less abundant but more potent splice variant compared to SDF-1α, were described. It was also shown that SDF-1β enhances in vitro mineralization and increases mRNA and protein levels of key osteogenic markers during bone morphogenetic protein (BMP)-2-stimulated osteogenic differentiation of BMSCs.

The results presented in FIGS. 16A-F show that SDF-1β significantly potentiated Smad1/5/8-mediated BMP-2 signal transduction in genetically engineered BMSCs via Erk1/2 phosphorylation (p<0.05). Pretreatment with the CXCR4 antagonist AMD3100 or the specific MEK1/2 inhibitor U0126 abolished this effect. SDF-1β, independent of SDF-1α, significantly promoted the migratory response of CXCR4-expressing Jurkat cells and BMSCs (p<0.01).

SDF-1β also mediated significant apoptosis-resistance in genetically engineered BMSCs (p<0.001). The greater number of surviving cells was found to be a result of enhanced autophagy.

In summary, these data indicate that SDF-1β may exert its biological activities during osteogenic differentiation of BMSCs in both an autocrine and paracrine fashion.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of increasing autophagy in a cell comprising contacting the cell with an effective amount of a composition comprising an agent that increases the bioavailability of an active form of SDF-1 to increase autophagy in the cell.

2. The method of claim 1 wherein the agent is an active isoform of SDF-1.

3. The method of claim 2 wherein the active form of SDF-1 is a polypeptide comprising the amino acid sequence of SEQ ID NO:2, 3, 5, 7, 9, or 11.

4. The method of claim 3 wherein the polypeptide is a fusion protein.

5. The method of claim 1 wherein the agent is an vector comprising expression elements operably linked to a nucleic acid encoding a polypeptide comprising the amino acid sequence of SEQ ID NO:1, 2, 3, 4, 5, 6, 7, 8, 9, or 10.

6. The method of claim 1 wherein the agent is mRNA encoding a polypeptide comprising the amino acid sequence of SEQ ID NO:1, 2, 3, 4, 5, 6, 7, 8, 9, or 10.

7. The method of claim 1 wherein the agent is a small molecule.

8. The method of claim 7 wherein the small molecule is metformin.

9. The method of claim 1 wherein the agent is a transcription factor that increases expression of SDF-1.

10. The method of claim 1 wherein the agent is a functional nucleic acid that reduces or inhibits the expression or activity of an miRNA that targets SDF-1 mRNA.

11. The method of claim 10 wherein the miRNA is selected from the group consisting of miRs 29a-5p, 1244, 141, 144, 200a, or 200c.

12. The method of claim 1 wherein the agent decreases expression or production of inactive or antagonistic forms of SDF-1.

13. The method of claim 12 wherein the agent is a small molecule.

14. The method of claim 12 wherein the agent is an inhibitor of a metalloproteinase, CD26/dipeptidyl peptidase IV (DPP4), a serine protease, or a leukocyte elastase.

15. The method of claim 14 wherein the agent is an inhibitor of DPP4.

16. The method of claim 15 wherein the inhibitor is selected from the group consisting of sitagliptin, vildagliptin, saxagliptin, linagliptin, dutogliptin, gemigliptin, alogliptin, and pharmaceutically acceptable salts, or active analogs thereof.

17. The method of claim 15 wherein the inhibitor of DPP4 is an miRNA.

18. The method of claim 17 wherein the miRNA is miR-3173-5p.

19. The method of claim 1 wherein the agent increases expression of a SDF-1 receptor.

20. A method of treating symptom of aging comprising increasing the autophagy of cells in and around the injury according to the method of claim 1 in an amount effective to one or more symptoms associated with aging.


SEQUENCE LISTING (15 sequences)

SEQ ID NO: 1 - protein, 93 aa, Human
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 2 - protein, 72 aa, Human
KPVSLSYRCPCRFFESHVARANVKHLKILNTPNCALQIVARLKNNNRQVCIDPKLKWIQEYLEKALNKRFKM

SEQ ID NO: 3 - protein, 68 aa, Human
KPVSLSYRCPCRFFESHVARANVKHLKILNTPNCALQIVARLKNNNRQVCIDPKLKWIQEYLEKALNK

SEQ ID NO: 4 - protein, 119 aa, Human
MNAKVVVVLVLVLTALCLSDGKPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 5 - protein, 98 aa, Human
KPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 6 - protein, 140 aa, Human
MNAKVVVVLVLVLTALCLSDGKPVSLSYRCPCRFFESHVARANVKHLKILNTPNCALQIVARLKNNNRQVCIDPKLKWIQEYLEKALNNLISAAPAGKRVIAGARALHPSPPRACPTARALCEIRLWPPPEWSWPSPGDV

SEQ ID NO: 7 - protein, 119 aa, Human
KPVSLSYRCPCRFFESHVARANVKHLKILNTPNCALQIVARLKNNNRQVCIDPKLKWIQEYLEKALNNLISAAPAGKRVIAGARALHPSPPRACPTARALCEIRLWPPPEWSWPSPGDV

SEQ ID NO: 8 - protein, 90 aa, Human
MNAKVVVVLVLVLTALCLSDGKPVSLSYRCPCRFFESHVARANVKHLKILNTPNCALQIVARLKNNNRQVCIDPKLKWIQEYLEKALNNC

SEQ ID NO: 9 - protein, 69 aa, Human
KPVSLSYRCPCRFFESHVARANVKHLKILNTPNCALQIVARLKNNNRQVCIDPKLKWIQEYLEKALNNC

SEQ ID NO: 10 - protein, 100 aa, Human
MNAKVVVVLVLVLTALCLSDGKPVSLSYRCPCRFFESHVARANVKHLKILNTPNCALQIVARLKNNNRQVCIDPKLKWIQEYLEKALNKIWLYGNAETSR

SEQ ID NO: 11 - protein, 79 aa, Human
KPVSLSYRCPCRFFESHVARANVKHLKILNTPNCALQIVARLKNNNRQVCIDPKLKWIQEYLEKALNKIWLYGNAETSR

SEQ ID NO: 12 - protein, 6 aa, Artificial Sequence
  Peptide
GSGSGS

SEQ ID NO: 13 - protein, 6 aa, Artificial Sequence
  Peptide
ASGGGS

SEQ ID NO: 14 - protein, 15 aa, Artificial Sequence
  Peptide
GGGGSGGGGSGGGGS

SEQ ID NO: 15 - protein, 20 aa, Artificial Sequence
  Peptide
GGGGSGGGGSGGGGSGGGGS